(19) United States
(12) Patent Application Publication
(10) Pub. No.: US 2002/0015941 A1
(43) Pub. Date: Feb. 7, 2002

(54) Method for treatment of neurodegenerative diseases

(75) Inventors: Tae-Wan Kim, Fort Lee, NJ (US); Rudolph E. Tanzi, Hull, MA (US); Andrew S. Yoo, Fort Lee, NJ (US)

Correspondence Address: STERNE, KESSLER, GOLDSTEIN AND FOX, LLP, 1100 NEW YORK AVENUE, N.W., SUITE 600, WASHINGTON, DC, 20005-3934, US

(73) Assignee: The General Hospital Corporation

(21) Appl. No.: 09/814,179

(22) Filed: Mar. 22, 2001

Related U.S. Application Data
(63) Continuation of application No. PCT/US00/20138, filed on Jul. 25, 2000.
(60) Provisional application No. 60/191,109, filed on Mar. 22, 2000.

(51) Int. Cl. A01N 37/18; A61K 38/00; C12Q 1/00

(52) U.S. Cl. 435/4; 514/2

(57) ABSTRACT

The invention relates to a method of identifying an agent useful in treatment of a neurodegenerative disease by assaying for capacitative calcium entry in cells treated with the agent. The invention also relates to a method of identifying an agent which inhibits capacitative calcium entry-linked γ-secretase activity by assaying for capacitative calcium entry in cells treated with the agent. The invention is further related to a method of treatment of a neurodegenerative disease by administering an agent which is capable of potentiating capacitative calcium entry activity.

This application is a continuation of International Appl. No. PCT/US00/20138 having an International Filing Date of Jul. 25, 2000, and claims benefit of the earlier filing date of U.S. Appl. No. 60/191,109, filed Mar. 22, 2000, the contents of both which are herein incorporated by reference in their entirety.

### BACKGROUND OF THE INVENTION

1. Field of the Invention

The present invention is directed to a method of identifying an agent useful in treatment of neurodegenerative diseases by assaying for capacitative calcium entry in cells treated with the agent. The present invention is also directed to a method of identifying an agent which inhibits capacitative calcium entry-linked γ-secretase activity by assaying for capacitative calcium entry in cells treated with the agent. The invention is further directed to a method of treatment of neurodegenerative diseases by administering an agent which is capable of potentiating capacitative calcium entry activity.

2. Related Art

Inherited mutations in the genes encoding two homologous proteins, presenilins 1 and 2 (PS1 and PS2), account for up to 40% of the early-onset cases of familial Alzheimer's disease (FAD) (reviewed in Tanzi, R. E., J. Clin. Invest. 104:1175 (1999); Tanzi, R. E., et al., Neurobiol. Dis. 3:159 (1996)). Both PS1 and PS2 are polytopic membrane proteins containing eight putative transmembrane (TM) domains (Doan, A., et al., Neuron 17:1023 (1996); Li, X. and Greenwald, I., Proc. Natl. Acad. Sci. USA 95:7109 (1998)) and localized to intracellular membranes (Kovacs, D. M., et al., Nature Med. 2:224 (1996); Cook, D. G., et al., Proc. Natl. Acad. Sci. USA 93:9223 (1996); Kim, S. H., et al., Neurobiol. Dis. 7:99 (2000)). Although a majority of nascent full-length presenilins are rapidly degraded by proteasomes (Kim, T. -W., et al., J. Biol. Chem. 272:11006 (1997)), a subset of the presenilins are stabilized and undergo regulated endoproteolysis (Thinakaran, G., et al., Neuron 17:181 (1996); Thinakaran, G., et al., Neurobiol. Dis. 4:438 (1998); Kim, T. -W., et al., J. Biol. Chem. 272:11006 (1997)), yielding N- and C-terminal heterodimeric complexes (Seeger, M., et al., Proc. Natl. Acad. Sci. USA 94:5090 (1997); Capell, A., et al., J. Biol. Chem. 273:3205 (1998)) which comprise functional units of the presenilins (Saura, C. A., et al., J. Biol. Chem. 274:13818 (1999); Tomita, T., et al., J. Neurosci. 19:10627 (1999)).

The presenilins appear to play an essential role in the proteolytic processing of the amyloid β-protein precursor (APP) (i.e., γ-secretase cleavage) (De Strooper, B., et al, Nature 391:387 (1998); Wolfe, M. S., et al., Nature 398:513 (1999)) and in the trafficking and maturation of various cellular proteins, including Notch, TrkB, APLP2, and hIre1α (Annaert, W., and De Strooper, B., Trends Neurosci. 22:439 (1999); Naruse, S., et al., Neuron 21:1213 (1998); De Strooper, B., et al., Nature 398:518 (1999); Naruse, S., et al., Neuron 21:1213 (1998); Niwa, M., et al., Cell 99:691 (1999); Struhl, G., and Greenwald, I., Nature 398:522 (1999); Ye, Y., et al., Nature 398:525 (1999); Steiner, H., et al., J. Biol. Chem. 274:28669 (1999)). It has been demonstrated that two TM aspartate residues (D257 and D385 in PS1; D263 and D366 in PS2) are individually critical for presenilin-associated γ-secretase activity as well as presenilin endoproteolysis (Wolfe, M. S., et al., Nature 398:513 (1999); Steiner, H., et al., J. Biol. Chem. 274:28669 (1999); Kimberly, W. T., et al., J. Biol. Chem. 275:3173 (2000)). FAD-associated mutations in PS1 or PS2 give rise to an increased production of the 42-amino acid version of amyloid β-peptide (Aβ42) in AD patients (Scheuner, D., et al., Nat. Med. 2:864 (1996)) as well as transfected cell lines and transgenic animals expressing FAD mutant forms of PS1 or PS2 (Borchelt, D. R., et al., Neuron 17:1005 (1996); Citron, M., et al., Nature Med. 3:67 (1996); Duff, K., et al., Nature 383:710 (1996); Tomita, T., et al., Proc. Natl. Acad. Sci. USA 94:2025 (1997); Oyama, F., et al., J. Neurochem. 71:313 (1998)). Aβ42 is an initial species that are deposited into senile plaques (Iwatsubo, T., et al., Neuron 13:45 (1994)) and aggregates more readily than Aβ40 (reviewed in Selkoe, D. J., Trens Cell. Biol. 8:447 (1998)).

In addition, cells expressing FAD-linked variants of PS1 or PS2 exhibit an increased sensitivity to agonist-induced transient Ca²⁺ release (Guo, Q., et al., Neuroreport 8:379 (1996); Mattson, M. P.,et al., J. Neurochem. 70:1 (1998); Gibson, G. E., et al., Neurobiol. Aging. 18:573 (1997); Etcheberrigaray, R., et al., Neurobiol. Dis. 5:37 (1998)). However, a molecular connection between this Ca²⁺-related phenotype and other molecular consequences commonly associated with presenilin FAD, such as the increased generation of Aβ42 (Scheuner, D., et al., Nature Med. 2:864-870 (1996); Borchelt, D. R., et al., Neuron 17:1005 (1996); Duff, K., et al., Nature 383:710 (1996); Citron, M., et al., Nature Med 3:67 (1996); Oyama, F., et al., J. Neurochem. 71:313 (1998); Tomita, T., et al., Proc. Natl. Acad. Sci. USA 94:2025 (1997)), remains unresolved.

The Aβ42-promoting effect of FAD mutant presenilins does not appear to be cell type-specific (Scheuner, D., et al., Nat. Med. 2:864 (1996); Xia, W., et al., J. Biol. Chem. 272:7977 (1997); Tomita, T., et al., Proc. Natl. Acad. Sci. USA 94:2025 (1997); Borchelt, D. R., et al., Neuron 17:1005 (1996); Duff, K., et al., Nature 383:710 (1996); Citron, M., et al., Nature Med 3:67 (1996); Oyama, F., et al., J. Neurochem. 71:313 (1998)).

Calcium regulation plays an important role in many cellular processes. In non-excitable mammalian cells, activation of phosphoinositide-specific phospholipase C (PLC) produces inositol 1,4,5-triphosphate (IP₃), which in turn causes the release of intracellular calcium from its storage pools in the endoplasmic reticulum. This results in a transient elevation of cytosolic free Ca²⁺, which is normally followed by a Ca²⁺ influx from the extracellular space. By refilling the pools, Ca²⁺ influx plays an important role in prolonging the Ca²⁺ signal, allowing for localized signaling, and maintaining Ca²⁺ oscillations (Berridge, M. J., Nature 361:315-325 (1993)).

Store-operated calcium influx, also known as capacitative calcium entry (CCE), serves as a prominent Ca²⁺-refilling mechanism in both electrically non-excitable and excitable cells, such as neurons (Putney, Jr., J. W., Cell Calcium 7:1 (1986); Putney, Jr., J. W., Cell Calcium 11:611 (1990); Berridge, M. J., Biochem. J. 312:1 (1995); Grudt, T. J., et al., Mol. Brain Res. 36:93 (1996); Li, H. -S., et al., Neuron 24:261 (1999); Clapham, D. E., Cell 80:259 (1995)). Depletion of intracellular Ca²⁺ stores triggers CCE through a putative mechanism involving protein and/or membrane trafficking (Yao, Y., et al., Cell 98:475 (1999); Patterson, R. L., et al., Cell 98:487 (1999)). CCE is directly coupled to the filling state of the internal Ca²⁺ stores (Waldron, R. T., et al., J. Biol. Chem. 97:6440 (1997); Hofer, A. M., et al., J. Cell Biol. 140:325 (1998)), and a number of cellular functions are influenced by changes in CCE, including chaperone activities, gene expression, and apoptotic cell death (Meldolesi, J. and Pozzan, T., TIBS 23:10 (1998); Jayadev, S., J. Biol. Chem. 274:8261 (1999); Bezprozvanny, I., et al., Nature 351:751 (1991); Krause, K. -H., and Michalak, M., Cell 88:439 (1997); Camacho, P. and Lechleiter, J. D., Cell 82:765 (1995)).

### SUMMARY OF THE INVENTION

The present invention is directed to a method of identifying an agent useful in treatment of a neurodegenerative disease by assaying for capacitative calcium entry in cells treated with the agent. The present invention is also directed to a method of identifying an agent which inhibits capacitative calcium entry-linked γ-secretase activity by assaying for capacitative calcium entry in cells treated with the agent. The invention is further directed to a method of treatment of a neurodegenerative disease by administering an agent which is capable of potentiating capacitative calcium entry activity.

### BRIEF DESCRIPTION OF THE FIGS.

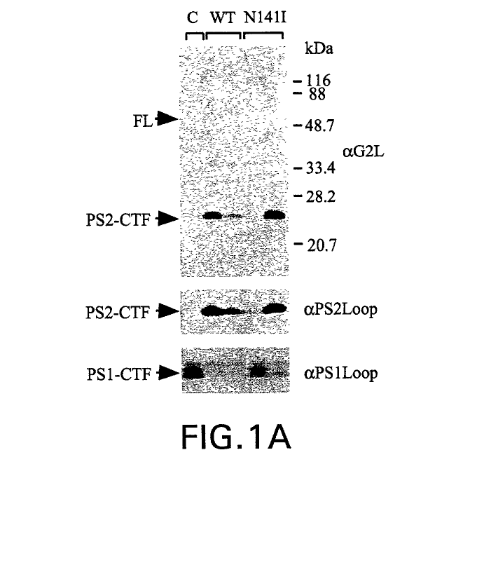

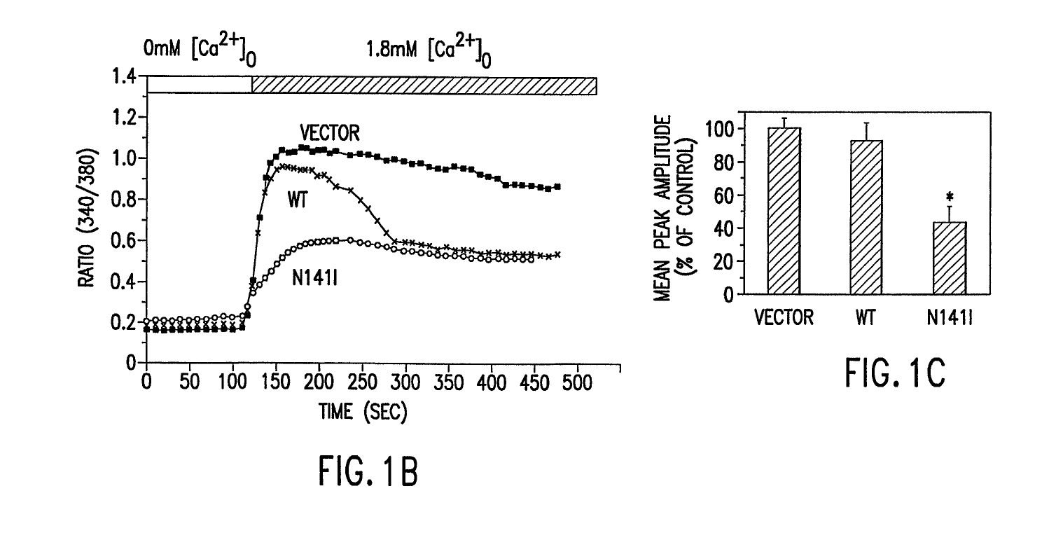

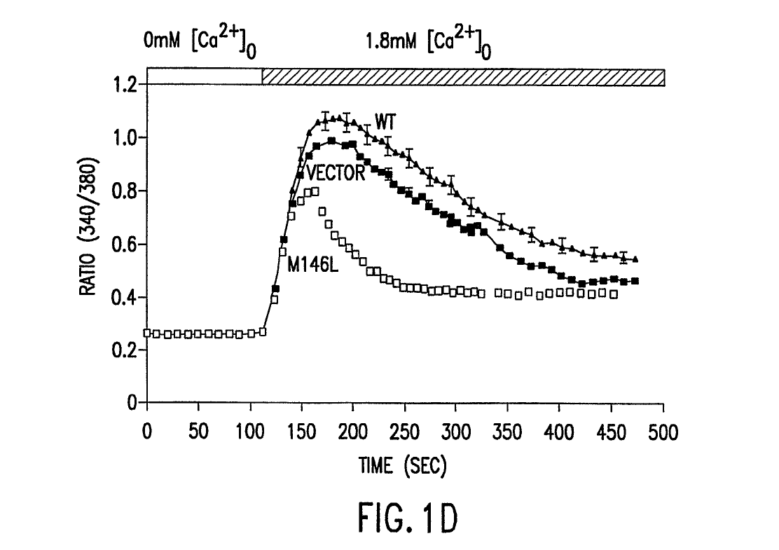

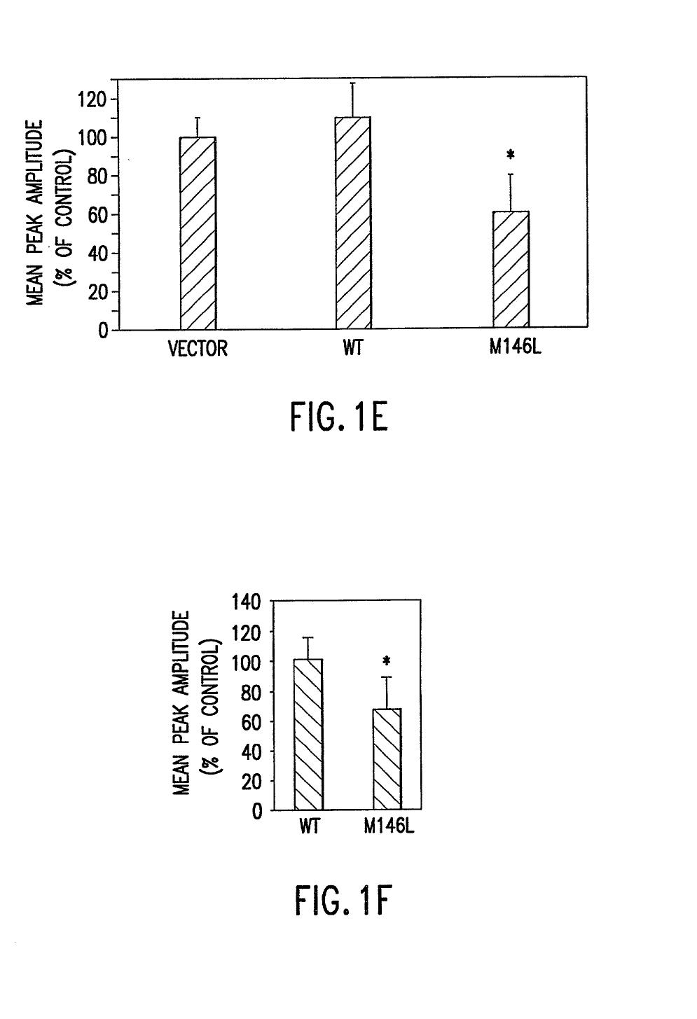

FIG. 1A-FIG. 1F. Attenuated capacitative Ca²⁺ entry (CCE) in cells expressing FAD mutant presenilins. FIG. 1A Lysates prepared from stable SY5Y cell lines expressing vector (c) and either wild-type (WT) or FAD mutant (N141I) forms of PS2 were analyzed by Western blotting using the PS antibodies indicated (Tomita, T., et al., Proc. Natl. Acad. Sci. USA 94:2025 (1997); Thinakaran, G., et al., Neuron 17:181 (1996)). Locations of full-length PS2 (FL) and C-terminal fragments of PS2 (PS2-CTF) and PS1 (PS1-CTF) are indicated by arrows. FIG. 1B Effect of the N141I PS2 FAD mutation on the CCE response. CCE was measured by ratiometric imaging in fura-2-loaded SY5Y cells stably transfected with vector, wild-type PS2 (WT), or mutant PS2 (N141I). Representative data from five independent experiments is shown (n=33). FIG. 1C Mean peak fluorescence amplitudes were calculated from five separate CCE-induction experiments, using SY5Y cells expressing vector, wild-type PS2 (WT), and N141I-PS2 (N141I) (*p<0.0001, compared to WT). FIG. 1D Effect of the M146L PS1 FAD mutation on the CCE response. CCE was measured by ratiometric imaging in fura-2-loaded SY5Y cells stably transfected with vector, wild-type PS1 (WT), or mutant PS1 (M146L) (n=26). FIG. 1E Mean peak fluorescence amplitudes were calculated from three independent CCE-induction experiments, using SY5Y cells expressing vector, wild-type PS1 (WT), and mutant PS1 (M146L) (*p<0.0001, compared to WT). Data points are mean fluorescence ratios (340 nm/380 mn)±S.E. (FIG. 1B, FIG. 1D), and columns are mean % increases±S.D. (FIG. 1C, FIG. 1E), as compared to vector-transfected cells. FIG. 1F Effect of the M146L PS1 FAD mutation on CCE in stable CHO cell lines. Mean peak fluorescence amplitudes were calculated from four independent CCE-induction experiments, using CHO cells stably expressing wild-type PS1 (WT) and mutant PS1 (M146L) (*p<0.0001, compared to WT). In each case, the wild-type and PS1-M146L clonal lines were paired for similar levels of expression. Data points are mean fluorescence ratios (340 nm/380 nm)±S.E. (A), and columns are mean % increases±S.D. (B, C).

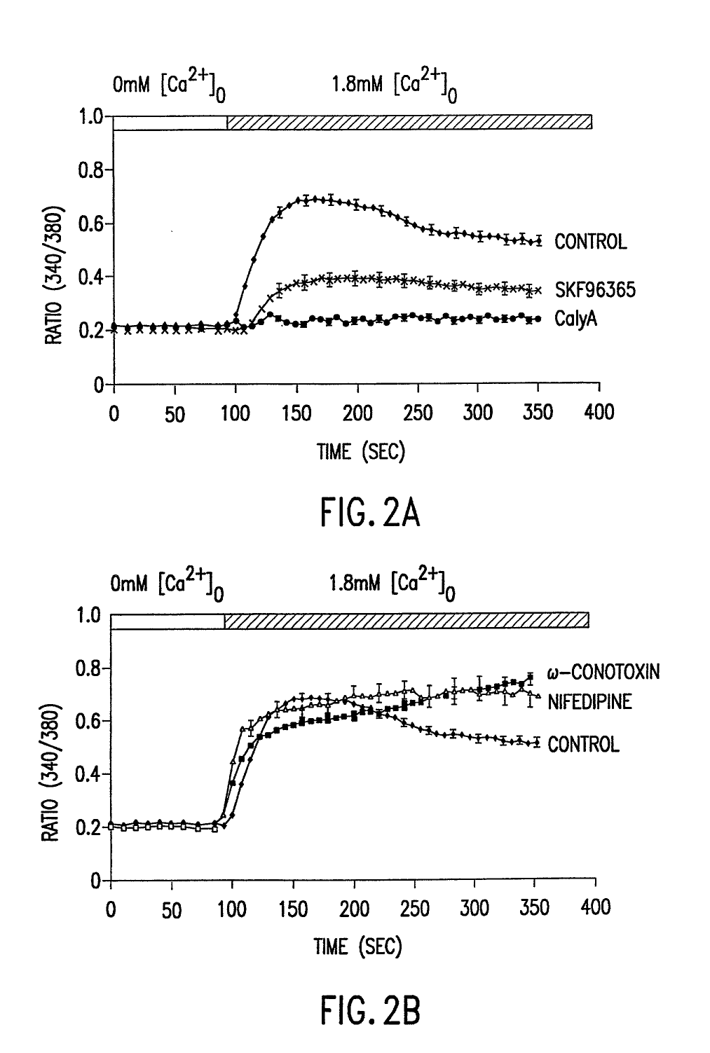

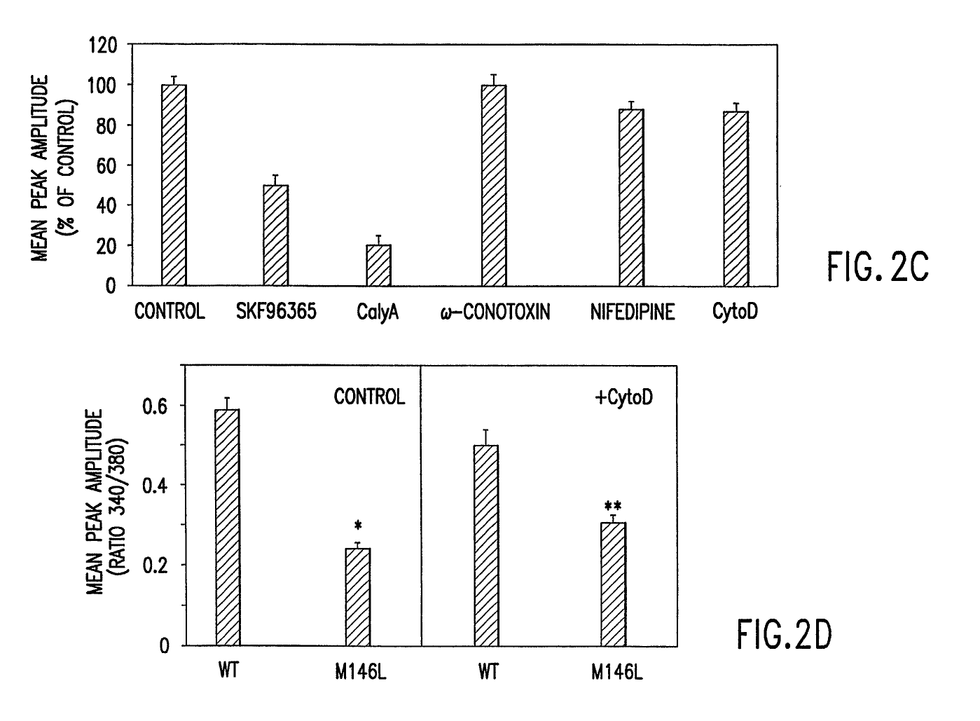

FIG. 2A-FIG. 2D. CCE-specific properties of the observed Ca²⁺ influx in SY5Y cell lines. FIG. 2A Inhibition of CCE by SKF96365 or Calyculin A (CalyA). SY5Y cells stably expressing wild-type PS2 were pretreated with either 100 μM SKF96365 for 1 hr or 100 nM CalyA for 20 min prior to induction of CCE. FIG. 2B Effects of L-type or N-type voltage-operated Ca²⁺ channel antagonists, nifedipine (1 μM) and ω-conotoxin GVIA (2 μM), respectively, on the CCE response in SY5Y cells. FIG. 2C Relative effects of SKF96365, CalyA, ω-conotoxin GVIA, nifedipine, and Cytochalasin D (CytoD) on CCE in wild-type PS2 cells. Columns are mean peak amplitudes±S.D., shown as % of control. FIG. 2D CytoD has no effect on the observed reduction in CCE caused by the M146L PS1 mutation. Mean peak amplitudes were determined from three independent experiments using SY5Y cells expressing wild-type PS1 (WT) or mutant PS1 (M146L), either without (Control) or with (+CytoD) a 2 hr pretreatment of 2 μM CytoD. Columns are mean peak amplitudes in fluorescence ratios±S.D. (*p<0.0001 and **p<0.001, respectively, as compared to WT).

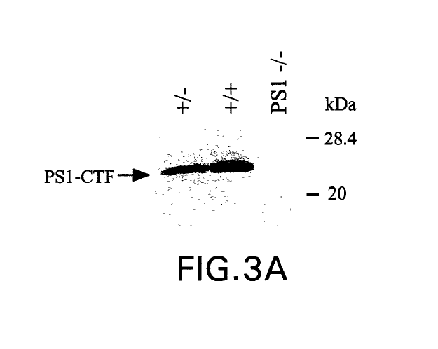

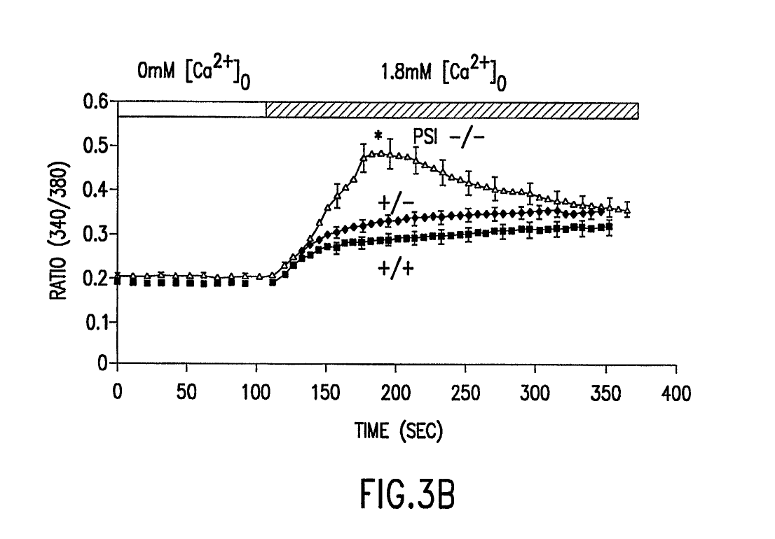

FIG. 3A-FIG. 3B. Potentiation of the CCE response by a PS1 deficiency. FIG. 3A Cultured cortical neurons from day 15.5 embryos from heterozygote (+/−, Control 1), homozygote (+/+, Control 2), or knock-out (−/−) mice were subjected to Western blotting using αPS1 Loop antibody (Thinakaran, G., et al., Neuron 17:181 (1996)). FIG. 3B CCE was greatly potentiated in PS1-deficient neurons (PS1−/−) as compared to control 1 (+/−) or control 2 (+/+). Data points are mean fluorescence ratios±S.E. in 27-34 cells (*p<0.0001, compared to controls). CCE was induced by incubating cells with Ca²⁻-free media containing 2 μM cyclopiazonic acid (CPA) for 30 minutes, then washing the cells with Ca²⁻-free HBSS (0 mM [Ca²⁺]₀; see Experimental Procedures), and replacing Ca²⁺-free buffer with Ca²⁺-containing media (1.8 mM [Ca²⁺]₀.

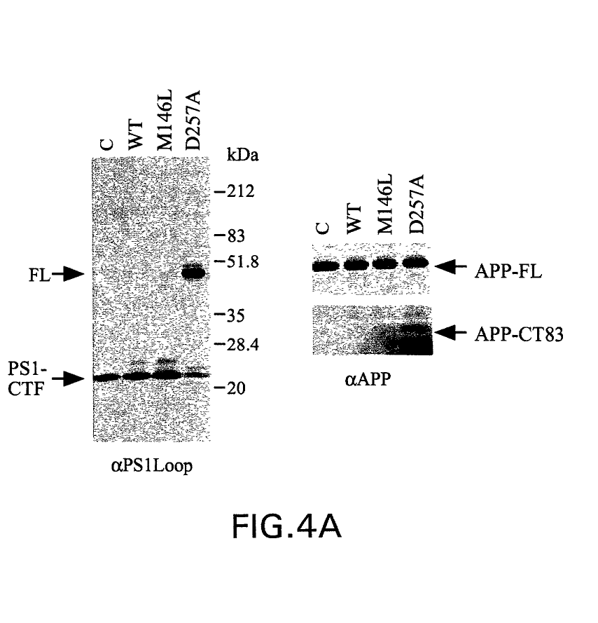

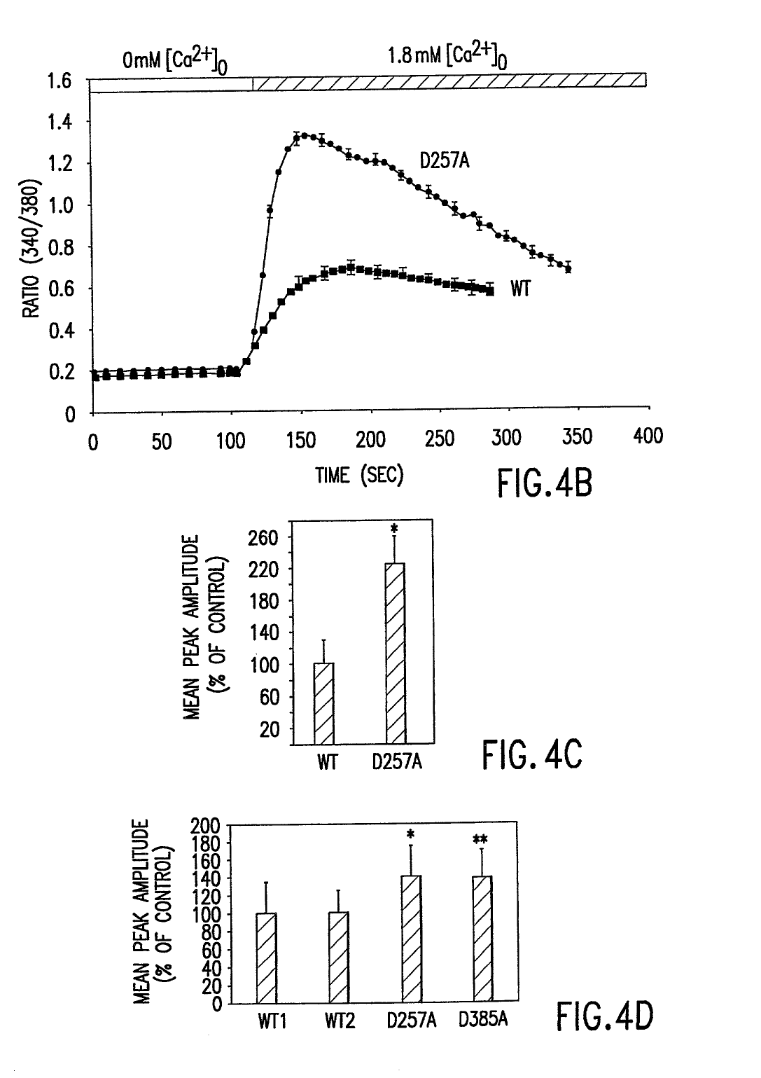

FIG. 4A-FIG. 4D. Potentiation of the CCE response by inactivation of PS1-associated γ-secretase activity. FIG. 4A Detergent lysates prepared from SY5Y cells stably transfected with vector (C), wild-type PS1 (WT), FAD mutant PS1 (M146L), or D257A-PS1 (D257A) were analyzed by Western blot analyses using αPS1 Loop antibody (left panel). Arrows denote full-length PS1 (FL) and endoproteolytic PS1 C-terminal fragments (PS1-CTF). An identical blot was probed with anti-APP antibody (C7) to detect APP holoprotein (APP-FL) as well as an endogenous APP C-terminal fragment (APP-CT83) (right panel). FIG. 4B Potentiation of the CCE response in SY5Y cells stably expressing D257A-PS1. Data points are mean fluorescence ratios±S.E. in 30 cells. FIG. 4C Mean peak fluorescence amplitudes were calculated from three independent CCE-induction experiments using SY5Y cells expressing wild-type PS1 (WT) or D257A-PS1 (D257A). Columns are mean peak amplitudes±S.D., shown as % of control (*p<0.0001, as compared to WT). FIG. 4D Mean peak fluorescence amplitudes were calculated from two independent CCE-induction experiments using four different clonal CHO cell lines expressing wild-type PS1 (WT1 and WT2), D257A-PS1 (D257A), or D385A-PS1 (D385A). Columns are mean peak amplitudes±S.D., shown as % of control (*p<0.0001, as compared to WT2; **p<0.0001, as compared to WT1).

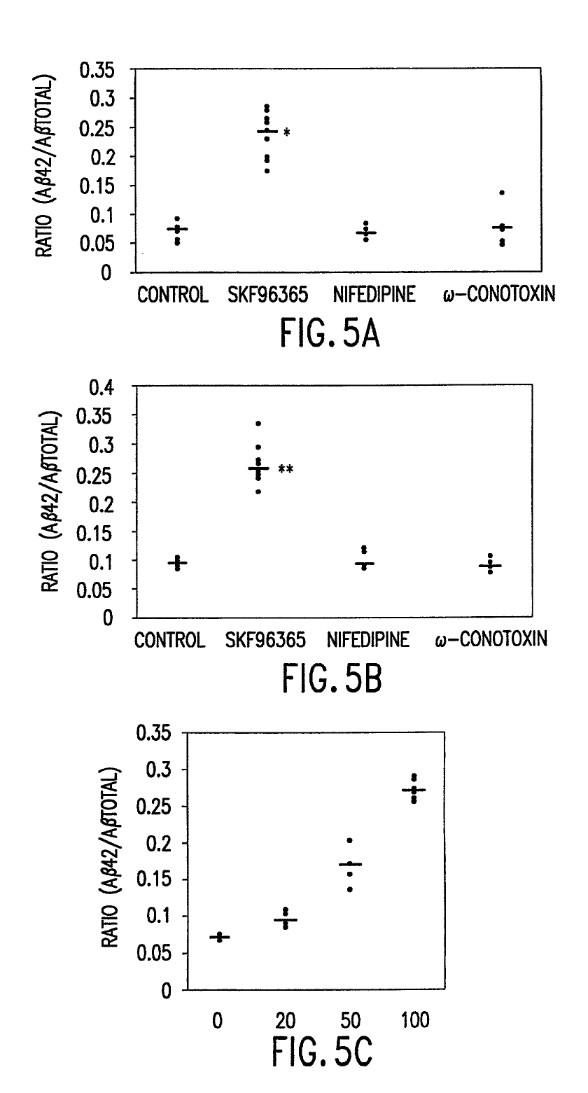

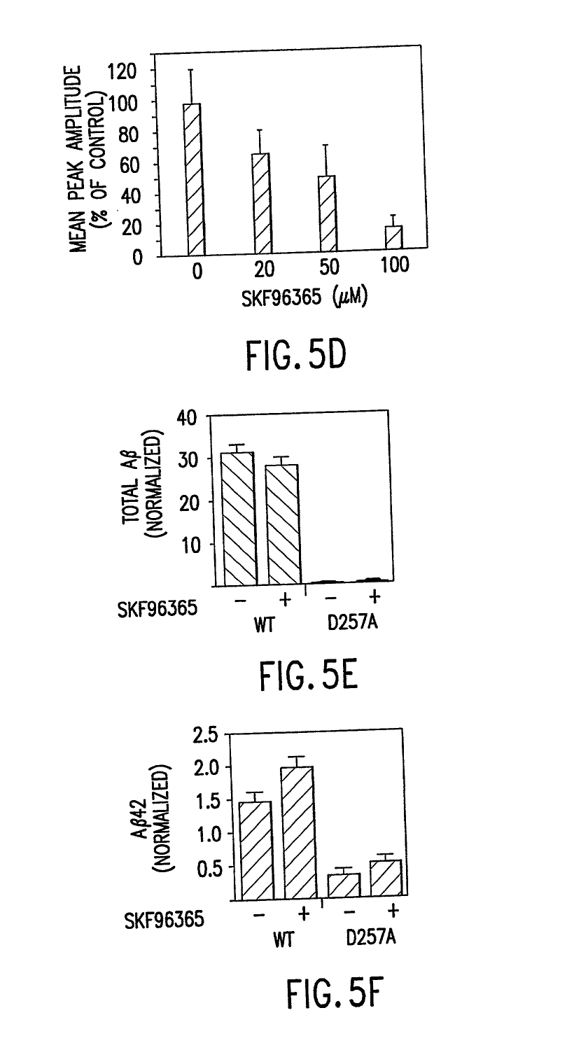

FIG. 5A-FIG. 5F. Effects of SKF96365 (100 μM), nifedipine (1 μM), and ω-conotoxin GVIA (1 μM) on the ratio of Aβ42/Aβtotal in CHO (FIG. 5A) or HEK293 (FIG. 5B) cells stably overexpressing human APP (12 hour treatment). Controls were DMSO (solvent) only. Amounts of Aβ42 and Aβtotal were determined by sandwich ELISA (Xia, X., et al., J. Biol. Chem. 2 72:7977 (1997)). The ratios of Aβ42/Aβtotal from three independent experiments were plotted. Horizontal bars represent average Aβ42 to Aβtotal ratios (n=12, *p<0.0001 and **p<0.0005, respectively, as compared to controls). Correlation of reduced CCE and increases in the Aβ42/Aβtotal ratio. CHO cells stably expressing human APP were treated with indicated concentrations of SKF9635 for 12 hours. Relative mean peak amplitudes (FIG. 5D) and corresponding Aβ42/Aβtotal ratios (FIG. 5C) are shown. CHO cells stably expressing APP and PS1 variants (either PS1 wild-type [WT] or D257A-PS1 [D257A]) were incubated in the absence (−) or presence (+) of 50 μM SKF96365. Columns represents relative amounts of total Aβ (FIG. 5E) or Aβ42 (FIG. 5F) in the culture media. All values were normalized to total protein amounts in the cell lysates.

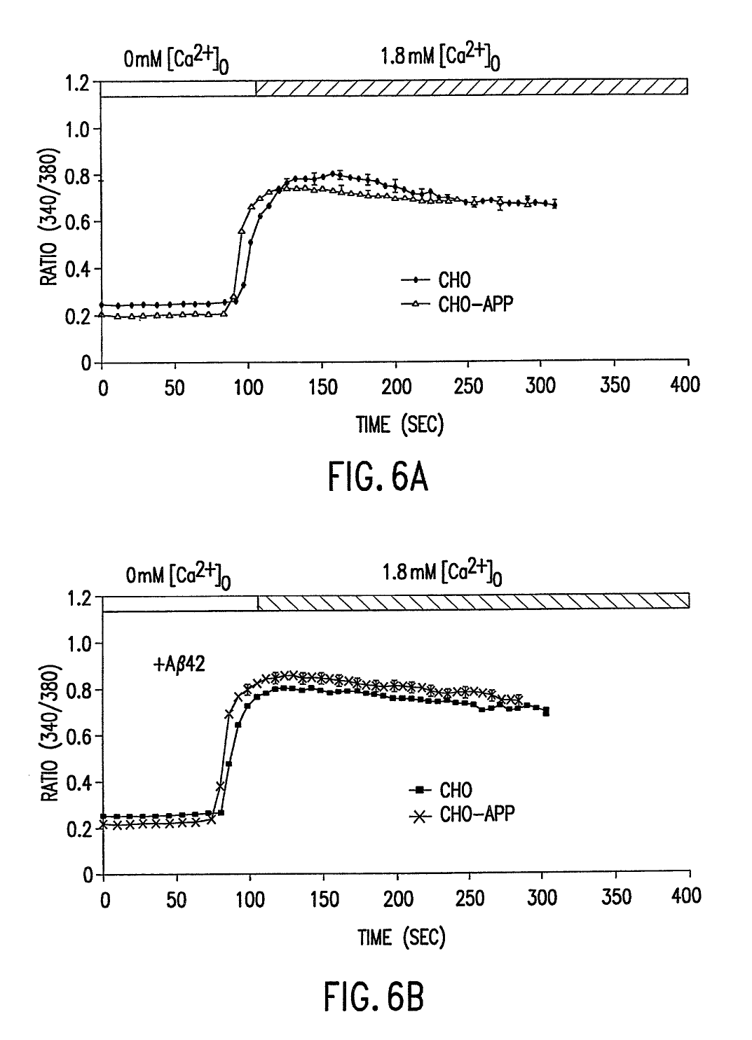

FIG. 6A-FIG. 6B. Effect of stable overexpression of human APP (FIG. 6A) and Aβ42 pretreatment (FIG. 6B) on the CCE response in CHO cells. FIG. 6A CCE was assayed by ratiometric Ca²⁺ imaging using either native CHO cells (CHO) or CHO cells stably overexpressing human APP₆₉₅ (CHO-APP). FIG. 6B CHO and CHO-APP cells were pre-incubated with 20 PM Aβ42 for 3 hours prior to induction of CCE (compare to FIG. 6A). Data points are mean fluorescence ratios±S.E. in 33 cells.

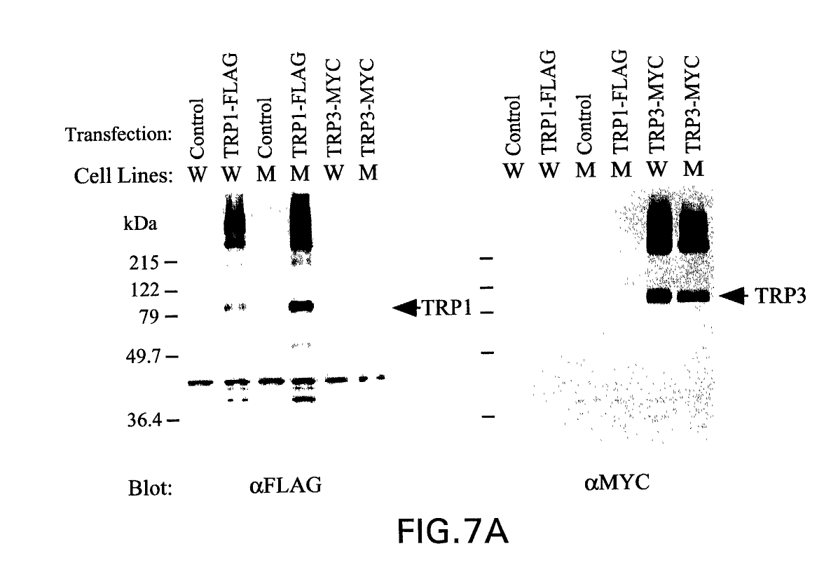

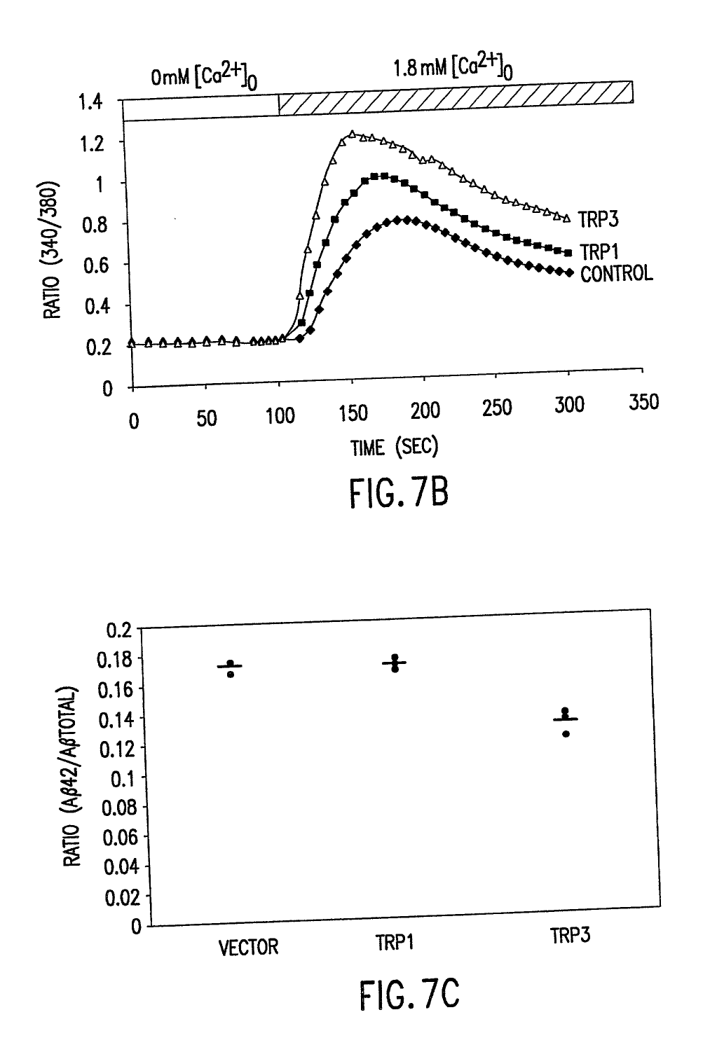

FIG. 7A. Expression of detection of TRP1 and TRP3 in CHO cells. Stable CHO cell lines expressing either wild-type PS1(W) or M146L mutant PS1 (M) were transiently transfected with empty vector (Control), FLAG-tagged TRP1 expression construct (TRP 1-FLAG), and MYC-tagged TRP3 expression construct (TRP3-MYC). The cell lysates were analyzed by Western blot analyses using anti-FLAG (left) or anti-MYC (right) antibodies. Expressed TRP1 and TRP3 are indicated by arrows.

FIG. 7B. Effect of overexpression of TRP1 and TRP3 on capacitative calcium entry (CCE) in stable CHO cells expressing M146L FAD mutant PS1. CCE was potentiated in both TRP1- and TRP3-transfected cells as compared to vector-transfected (Control) cells, but to greater extent in TRP3-expressing cells. The ratiometric calcium imaging was performed as described in the manuscript.

FIG. 7C. Effects of overexpression of vector, TRP1, and TRP3 on the ratio of Aβ42/Aβtotal in CHO cells stably expressing M146L mutant PS1. Amounts of Aβ42 and Aβtotal were determined by sandwich ELISA.

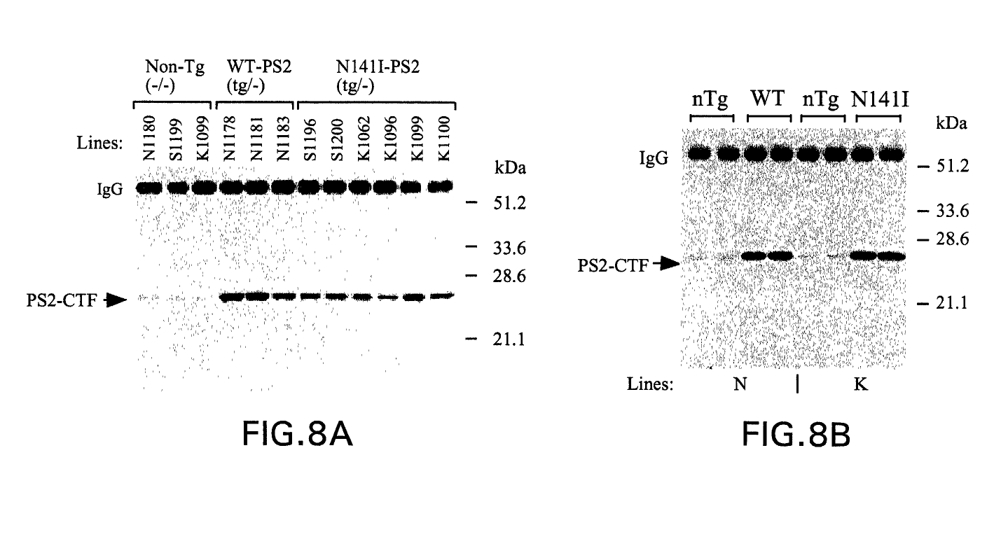

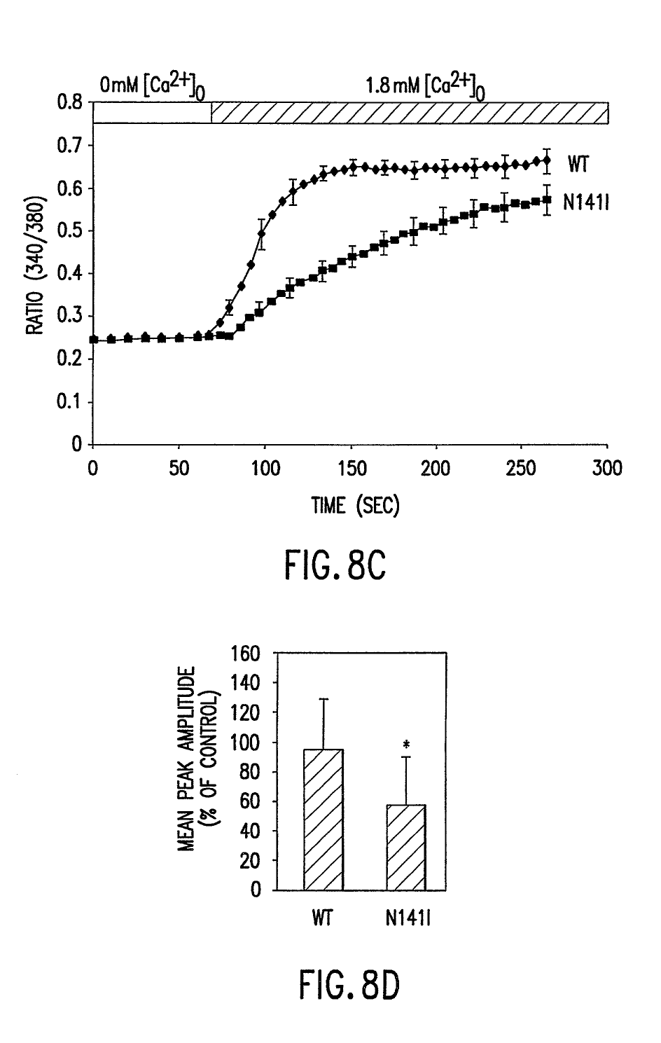

FIG. 8A-FIG. 8D. Primary Cortical Neurons Derived from N141I-PS2 Transgenic Mice Exhibit Attenuated CCE. FIG. 8A Characterization of PS2 in transgenic mice. Immunoprecipitation-Western blotting analysis was performed using αPS2loop in the lysates prepared from brain tissues of transgenic mice expressing a construct encoding either wild-type (WT-PS2) or N141I FAD mutant (N141I-PS2) PS2, along with non-transgenic samples (Non-Tg). FIG. 8B Lines with similar levels of protein expression were paired among N and K lines and protein extracts were analyzed by Immunoprecipitation-Western blotting analysis. Representative blot is shown. FIG. 8C Effects of the N141I-PS2 mutation on CCE in cultured cortical neurons from day 18.5 embryos. FIG. 8D Average mean peak amplitudes were shown as mean fluorescence ratios (340 nm/380 nm)±S.D. (n=˜50; *p<0.0001, compared to WT).

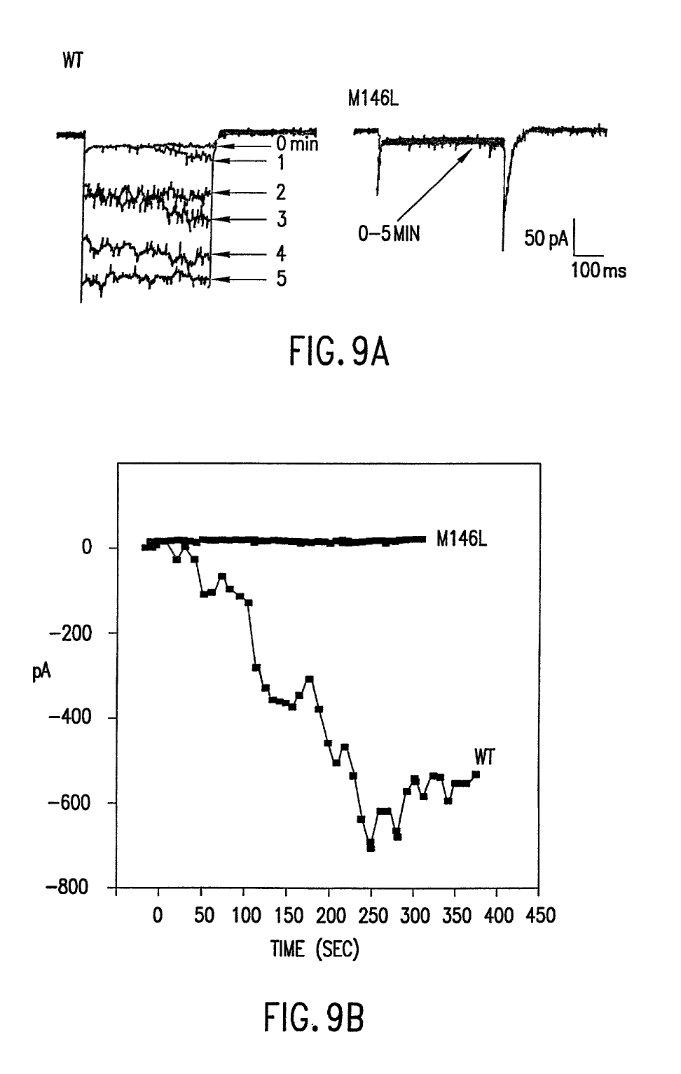

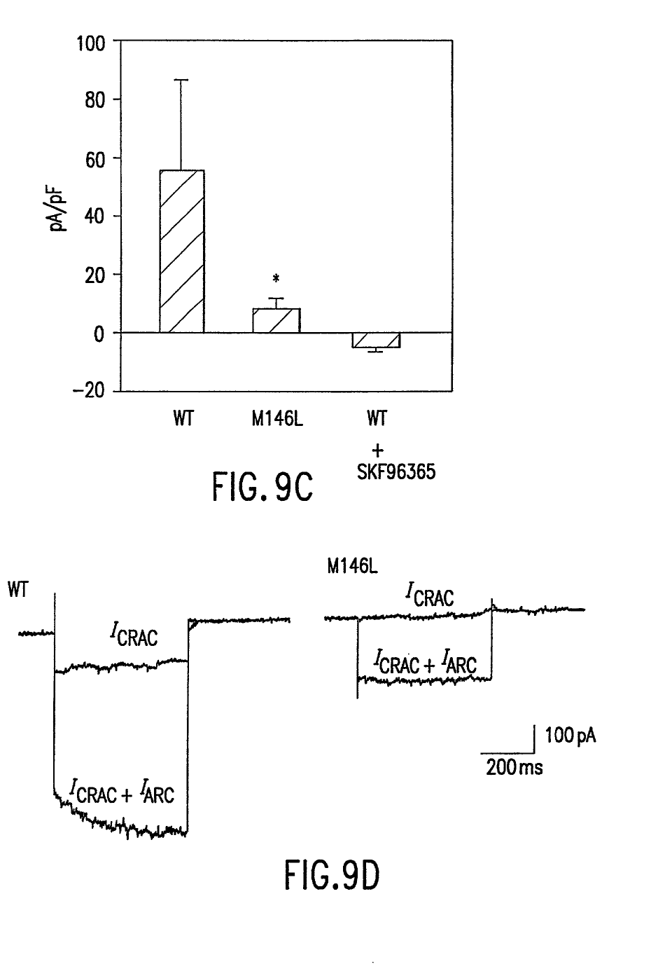

FIG. 9A-FIG. 9D. Impaired Calcium Release-Activated Calcium Currents (I_{CRAC}) in M146L-PS1 Cells. FIG. 9A I_{CRAC} channel activities were measured in the stable CHO cells expressing either wild-type (WT) or FAD mutant (M146L) PS1 by the whole-cell patch clamp experiments. The currents were activated following dialysis with 10 mM BAPTA (passive depletion). Membrane potential was held at 0 mV, and hyperpolarizing voltage pulses at −120 mV were applied every 10 s. The transient and leak currents were not canceled. FIG. 9B Comparison of time courses of the activation of I_{CRAC} channels in wild-type and M146L PS1 cells. Inward currents were evoked by applying hyperpolarizing pulse at 120 mV at a holding potential of 0 mV. Data points are the current levels measured at every 10 s. The leak currents were canceled. FIG. 9C Comparison of average peak I_{CRAC} current densities (pA/pF) from wild-type (WT) and M146L-PS1 cells. Wild-type PS1 cells were also pretreated in parallel with 10 μM SKF96365 for 30 min before the current measurement (WT+SKF96365). The average peak current density in M1465L-PS1 cells was significantly smaller than that of wild-type PS1 cells (n=23, *p<0.05). FIG. 9D Arachidonate-regulated Ca²⁺ currents (I_{ARC}) were preserved in M146L-PS1 cells. After I_{CRAC} currents reached the stable levels in 6-7 min, arachidonic acid (8 μM) were added to induce I_{ARC} currents on top of I_{CRAC} currents. Currents were measured as described in FIG. 9A.

### DETAILED DESCRIPTION OF THE PREFERRED EMBODIMENTS

As described in detail in the Example section below, it has been discovered that capacitative calcium entry (CCE) activity is reduced in the presence of presenilin familial Alzheimer's disease (FAD) mutations. Moreover, reduced CCE leads to increased production of Aβ42. To define further the mechanism underlying the enhanced CCE in PS1-deficient neurons, the effect of inhibition of PS1-associated γ-secretase activity on CCE was examined. It has been discovered that CCE activity is inversely correlated to presenilin-linked γ-secretase activity.

The present invention is directed to a method of identifying an agent useful in treatment of a neuro degenerative disease, the method comprising:

(a) assaying for capacitative calcium entry (CCE) activity in cells treated with an agent;

(b) assaying for CCE activity in cells untreated with the agent; and

(c) comparing the CCE activities of (a) and (b) to determine whether the agent potentiates CCE activity in the cells treated with the agent, thereby identifying an agent useful in treatment of neurodegenerative disease.

In the invention, the method can further comprise:

(d) assaying for CCE activity in cells treated with the agent, wherein the cells overexpress a transient receptor potential protein (TRP);

(e) assaying for CCE activity in cells treated with said agent, wherein the cells do not overexpress a TRP; and

(f) comparing the CCE activities of (d) and (e) to determine whether said agent potentiates CCE activity in said cells that overexpress a TRP.

The invention is further directed to a method of identifying an agent which inhibits capacitative calcium entry (CCE)-linked γ-secretase activity, the method comprising:

(a) assaying for CCE activity in cells treated with an agent;

(b) assaying for CCE activity in cells untreated with the agent; and

(c) comparing the CCE activities of (a) and (b) to determine whether the agent increases CCE activity in the cells treated with said agent, thereby identifying an agent which inhibits γ-secretase activity.

In the invention, the method can further comprise:

(d) assaying for CCE activity in cells treated with the agent, wherein the cells overexpress a transient receptor potential protein (TRP);

(e) assaying for CCE activity in cells treated with said agent, wherein the cells do not overexpress a TRP; and

(f) comparing the CCE activities of (d) and (e) to determine whether said agent potentiates CCE activity in said cells that overexpress a TRP.

As described herein, by “agent” is intended a protein, nucleic acid, carbohydrate, lipid or a small molecule. The type of compounds which can be screened according to the invention are unlimited.

Candidate agents that potentiate CCE activity include, but are not limited to, neurosteroids, compound screening libraries, brain-derived neurotrophic factor (BDNF) for TRP3 (Li et al., Neuron 24:261-273 (1999)) and membrane-permeable diacylglycerol analogs, including 1-oleoyl-2-acetyl-sn-glycerol(OAG) and 1,2-dioctanoyl-sn-glycerol (DOG), for TRP3 and TRP6. CCE response can also be regulated by cellular substances including, but not limited to, an unidentified diffusible messenger (CIF), inositol phosphates (IP₃ and IP₄), cyclic GMP, or by covalent modification by enzymes such as protein kinases, protein phosphatases, small GTPases and cytochrome P450. Maitotoxin can also stimulate CCE channels (Worley, J. F. et al., J. Biol. Chem. 269:32055-32058 (1994)). Agents that potentiate CCE activity can be identified by assaying for CCE activity as according to the present invention.

Exemplary compound screening libraries with high structural diversity include, but are not limited to, the following:

| Company | Number of Compounds |
|---|---|
| AsInEx | 100,000 |
| Chembridge | 100,000 |
| Maybridge Chemical Co. | 50,000 |
| Microsource Discovery | 18,000 |
| Timtec, Inc. | 30,000 |

Such screening libraries can be purchased and used to screen a diverse pool of compounds in the CCE-based assays. A structure database, such as “Available Chemical Directory-Screening Compounds” from MDL of over one million chemical compounds from various suppliers, can be licensed. Screening is guided by structure information about the target and would focus on refining the drug development qualities of lead compounds with regard to adequate blood-brain barrier penetration, sustained half-life in animals, acceptable metabolism, low toxicity and good toleration, and stability. These compounds will be optimized for potency, selectivity, and specificity, and then in parallel, be tested in animal studies as well as studies aimed at determining the actual mechanism of action prior to lead optimization.

Methods for assaying CCE activity include physiological detection methods, including, but not limited to, calcium imaging and electrophysiological measurements. Calcium imaging can be performed as described in Yoo, A. S. J. et al., Brain Res. 827:19 (1999). For example, cells are grown on 25 mm-round glass coverslips for at least 24 hours before measuring [Ca²⁺]₁. Fura-2/AM is dissolved in DMSO and further solubilized in Pluronic acid (0.08%), in HBSS (145 mM NaCl, 2.5 mM KCl, 1 mM MgCl₂, 20 mM HEPES, 10 mM glucose, and 1.8 mM CaCl₂) containing BSA (1%). When Ca²⁺-free medium is used, Ca²⁺ is replaced with 50 μM EGTA. Fura-2 acetoxymethyl ester (fura-2/AM) is loaded by incubation with HBSS containing fura-2/AM (5 μM) at 37° C. for 30 minutes. Fluorescence emission at 505 nm is monitored at 25° C. using a dual wavelength spectrofluorometer system with excitation at 340 and 380 nm. Ratios (fluorescence intensity at 340 nm/380 nm) are obtained from 8-frame averages of pixel intensities at each of the excitation wavelengths. Of course, these conditions can be varied for optimal calcium imaging.

Electrophysiology measurements, such as patch-clamp technique, can also be used to measure CCE activity (Hamill, O. P. et al., Pflugers Arch. 391:85-100 (1981); Hofmann, T. et al., Nature 397:259-263 (1999); Krause, E. et al., J. Biol. Chem. 274:36957-36962 (1999)). As described in Hofmann et al., the patch-clamp technique (Hamill, O. P. et al., Pflugers Arch. 391:85-100 (1981)) can be used in whole-cell, cell-attached and inside-out mode. Solution B1 contains (in mM) 140 sodium isothionate, 5 potassium gluconate, 1.8 calcium gluconate, 1 magnesium gluconate, 10 glucose and 10 HEPES; solution B2 contains 120 sodium isothionate, 5.87 calcium gluconate, 1 magnesium gluconate, 10 EGTA, 10 glucose and 10 HEPES; solution B3 contains 120 CsCl, 1.8 calcium gluconate, 1 magnesium gluconate, 10 glucose and 10 HEPES; solution B4 contains 140 NMDG isothionate, 5 EGTA, 10 glucose and 10 HEPES; solution 5B contains 120 sodium isothionate, 1 EGTA, 10 glucose and 10 HEPES; solution B6 contains 10 calcium gluconate, 130 NMDG isothionate, 10 glucose and 10 HEPES; solution B7 contains 120 CsCl, 1 EGTA, 10 glucose and 10 HEPES; pipette solution P1 contained 120 CsCl, 5.87 calcium gluconate, 1 magnesium gluconate, 10 EGTA and 10 HEPES. Solutions are buffered to pH 7.4. The osmolarity is adjusted to 290-310 mosM with mannitol. An agar bridge serves as the electrical connection between the bath and the signal ground. In whole-cell experiments, the access resistance is less than 10 MΩ and series resistance compensation is set to 65-85%. For fluctuation analysis (Neher, E. and Stevens, C. F., Annu. Rev. Biophy. Bioeng. 6:345-381 (1977)), no series resistance compensation is used. Reversal potentials (E) of currents were determined from currents recorded during voltage ramps. Measurements are corrected for liquid-junction potentials. Relative ion permeabilities for monovalent cations are calculated as described (Hille, B. IONIC CHANNELS OF EXCITABLE MEMBRANES (Sinauer, Sunderland, Mass., 1992). The

$$\frac{{P}_{Ca}}{{P}_{{N}_{2}}}$$

permeability ratio is calculated according to the equation

$$\frac{{P}_{Ca}}{{P}_{{N}_{2}}} = \{ [ Na + ] a \times exp ( - {FE}_{Na} / RT ) exp ( {FE}_{Ca} / RT )$$

(exp(FE_{Cα}/RT)+1)}/4[Ca²⁺]₀), where R, T, and F are the gas constant, absolute temperature and Faraday's constant, respectively. Bath solutions can contain one of the following cations: Na³⁰ (solution B5, E_{Na}=−10.2±1.5 mV,n=15), Ca²⁻ (solution B6, E_{Ca}=−13.4±4.15 mV,n =12), or Cs⁺ (solution B7, E_{Cs}=1.6±2.1 mV,n =3). Analysis is performed with pClamp 6 software (Axon Instruments). Channel activity is expressed as NP₀, calculated for consecutive 5-s intervals. In whole-cell experiments, data are filtered at 1 kHz; in single-channel experiments, data are filtered at 2.5 kHz. Bath solutions containing SAG, SUG, DOG, OAG and PtdIns (Berridge, M. J., Biochem. J. 312:1-11 (1995); Putney, Jr., J. W., Cell Calcium 7:1-12 (1986)) P₂ are sonicated for 5 min before use. All experiments are performed at room temperature (21-26° C.).

Alternatively, as described in Krause, E. et al., J. Biol. Chem. 274:36957-36962 (1999), patch-clamp experiments can be performed in a tight-seal, whole-cell configuration (Hamill, O. P. et al., Pflugers Arch. 391:85-100 (1981)) at room temperature (24±2° C.) in a standard bath solution containing (in mM) 140 NaCl, 4.7 KCl, 10 CaCl₂, 1 MgCl₂, 10 HEPES, 10 glucose, pH 7.4. BaCl₂ (0.6 mM) is added to inhibit potassium currents. Patch pipettes are manufactured from borosilicate glass capillaries and has resistance of 2-4 megohms when filled with a standard pipette buffer containing (in mM) 110 Ca⁺-glutamate, 15.5 NaCl, 1 MgCl₂, 10 HEPES, 10 1,2-bis (2-aminophenoxy)ethane-N,BAPTA, 0.5 Mg-ATP, 10 glucose adjusted to pH 7.2 with CaOH. CaCl₂ is added to obtain different free [Ca²⁺] as calculated with the free-ware software WINMAXC. The standard solution is termed “Ca²⁺-free” if no Ca²⁺ was added (Ca²⁺<0.1 nM). To check the [Ca²⁺] of the ready-made solutions a calcium calibration buffer kit (number C-3722, Molecular Probes) is used. Patch-clamp experiments are recorded with a computer-controlled EPC9 patch clamp amplifier (HEEA; Lambrecht, Germany). Cell capacitance and series resistance are calculated with the software-supported internal routines of the EPC9 and compensated before each experiment. Data are sampled at 1 kH on the computer hard disc after low pass filtering at 600 Hz. In the whole-cell experiments voltage ramps are applied every 4 s to the cells (−140mV to 100 mV, slope 1 V/s). Variations of the patch-clamp technique or other methods for determining the CCE activity of cells, which are routine in the art, can also be used in carrying out the present invention.

Cells that can be used to screen for agents useful in treatment of neurodegenerative diseases include, but are not limited to, SH-SY5Y and SK-N-SH (human neuroblastoma cell lines), CHO (Chinese hamster ovary cell line), 293 (human embryonic kidney cell line), and Neuro2A (mouse neuroblastoma cell line). These cell lines can be used to stably or transiently overexpress wild-type or neurodegenerative disease-linked mutations. Inactive forms of the presenilins can be expressed in some of these cell lines as well (e.g., SH-SY5Y and CHO). All parental cells can be obtained from American Type Culture Collection. Since the above-mentioned cell lines possess the properties of transformed cells (cancer-like), hTERT-RPE1 and hTERT-BJ1 (telomerase-immortalized human retinal pigment epithelial cell lines) can also be used (both commercially available from Clontech), which grow continuously without transformed phenotype. Additional cells types that can be used in the invention include mouse skin fibroblasts, cultured embryonic primary neurons, and any other cells derived from transgenic mice expressing wild-type (WT-PS1 or WT-PS2) or FAD mutants (e.g., M146L-PS1 or N141I-PS2) of human presenilins, human skin fibroblasts derived from patients carrying FAD-causing presenilin mutations, mouse skin fibroblasts, cultured embryonic primary neurons, and any other cells derived from PS1-knock out transgenic mice (containing null mutation in the PS1 gene). Other cell types are readily known to those of ordinary skill in the art.

In the invention, the agent can be tested in cells having “neurodegenerative disease-linked mutations,” i.e., cells expressing genes that carry mutations causative of neurodegenerative diseases such as, but not limited to, Alzheimer's disease (AD), Parkinson's disease, Huntington's disease, and amyotrophic lateral sclerosis (ALS). Preferred cells to be tested are cells having AD-linked mutations. Mutations causative of AD include AD-linked familial mutations, genetically associated AD polymorphisms, and sporadic AD. AD-linked familial mutations include AD-linked presenilin mutations (Cruts, M. and Van Broeckhoven, C., Hum. Mutat. 11:183-190 (1998); Dermaut, B. et al., Am. J. Hum. Genet. 64:290-292 (1999)), and amyloid β-protein precursor (APP) mutations (Suzuki, N. et al., Science 264:1336-1340 (1994); De Jonghe, C. et al., Neurobiol. Dis. 5:281-286 (1998)). Genetically associated AD polymorphisms include, but are not limited to, polymorphisms such as apolipoprotein E (ApoE) mutations (e.g., APOE-ε4) (Strittmatter, W. J. et al., Proc. Natl. Acad. Sci. USA 90:1977-1981 (1993)). As used herein, a DNA polymorphism is intended a variation in the genome having a prevalence of greater than about 10%.

Mutations causative of Parkinson's include, but are not limited to, mutations in synuclein and parkin. Mutations causative of Huntington's include, but are not limited to, Huntingtin with a triplet (CHE) repeat expansion. Mutations causative of ALS include, but are not limited to, mutations in superoxide dismutase-1 gene.

More specifically, such cells can include, but not limited to, one or more of the following mutations, for use in the invention: APP FAD mutations (e.g., E693Q (Levy E. et al., Science 248:1124-1126 (1990)), V717 I (Goate A. M. et al., Nature 349:704-706 (1991)), V717F (Murrell, J. et al., Science 254:97-99 (1991)), V717G Chartier-Harlin, M. C. et al., Nature 353:844-846 (1991)), A682G (Hendriks, L. et al., Nat. Genet. 1:218-221 (1992)), K/M670/671N/L (Mullan, M. et al Nat. Genet. 1:345-347 (1992)), A713V (Carter, D. A. et al., Nat. Genet. 2:255-256 (1992)), A713T (Jones, C. T. et al., Nat. Genet. 1:306-309 (1992)), E693G (Kamino, K. et al., Am. J. Hum. Genet. 51:998-1014 (1992)), T673A (Peacock, M. L. et al., Neurology 43:1254-1256 (1993)), N665D (Peacock, M. L. et al., Ann. Neurol. 35:432-438 (1994)), I 716V (Eckman, C. B. et al., Hum. Mol. Genet. 6:2087-2089 (1997)), and V715M (Ancolio, K. et al., Proc. Natl. Acad. Sci. USA 96:4119-4124 (1999))); presenilin FAD mutations (e.g., all point (missense) mutations except one - - - 113Δ4 (deletion mutation)); PS1 mutations (e.g., A79V, V82L, V96F, 113Δ4 , Y115C, Y115H, T116N, P117L, E120D, E120K, E123K, N135D, M139, I M139T, M139V,I 143F, 1143T, M461, I M146L, M146V, H163R, H163Y, S169P, S169L, L171P, E184D, G209V, I213T, L219P, A231T, A231V, M233T, L235P, A246E, L250S, A260V, L262F, C263R, P264L, P267S, R269G, R269H, E273A, R278T, E280A, E280G, L282R, A285V, L286V, S290C (Δ9), E318G, G378E, G384A, L392V, C410Y, L424R, A426P, P436S, P436Q); PS2 mutations (R62H, N141I, V148I, M293V); and genetic risk factors. For example, APOE4 inheritance of apoE4 allele (vs. apoE3 or apoE2) confers greater risk to develop Alzheimer's disease in late life. By the present invention, it can be tested whether treatment of cells with a particular apoe type or overexpressing a particular apoe would affect CCE activity. For example, if apoE4 reduces CCE, agents can be identified which enhance the CCE in the presence of apoE4 by monitoring CCE changes following treatment with compounds.

The transient receptor potential protein (TRP) is a protein believed to mediate the CCE in the plasma membrane of mammalian cells. Seven different human TRPs (cDNAs) have been described (TRP1, TRP2, TRP3, TRP4, TRP5, TRP6, TRP7) and all exhibit different developmental and tissue distributions (reviewed in Philipp, S. et al., “Molecular Biology of Calcium Channels in Calcium Signaling,” in:CRC METHODS IN SIGNAL TRANSDUCTION, pp.321-342, Putney, Jr., J. W., et al., eds. (2000); Birnbaumer, L. et al., Proc. Natl. Acad. Sci. USA 93:15195-15202 (1996)). Thus, in the invention, the cells can overexpress one or more TRPs (Birnbaumer, L. et al., Proc. Natl. Acad. Sci. USA 93:15195-15202 (1996); Li, H. -S. et al. Neuron 24:261-273 (1999); U.S. Pat. No. 5,923,417). According to the invention, the agent can, for example, regulate expression of TRP in a cell having the neurodegenerative disease-linked mutation, increase TRP targeting, or regulate cellular maturation of TRP. Cellular maturation of TRP can be regulated by, for example, increasing the level of functional TRP or decreasing degradation of functional TRP. Functional TRP is a subpopulation of TRP that target to the surface or cellular locus where TRP functions, e.g., plasma membrane.

For stable or transient overexpression of TRPs or neurodegenerative disease-linked mutants, cDNAs coding for different neurodegenerative disease-linked mutants or different TRPs can be transfected either transiently or stably transfected using methods well known in the art, for example, Superfect transfection reagent (Qiagen).

In addition, the agent of interest can be tested in parental cells and/or wild-type cells as control.

Agents which enhance CCE activity can be used to treat subjects predisposed to or having a neurodegenerative disease.

Thus, the invention is directed to a method of treatment of a neurodegenerative disease in a subject, the method comprising:administering to said subject a pharmaceutically effective amount of an agent capable of potentiating capacitative calcium entry (CCE) activity in said subject. The treatment can provide prevention of a neurodegenerative disease in a subject predisposed to the neurodegenerative disease. The treatment can provide therapy of a neurodegenerative disease in a subject in need thereof. In the invention, neurodegenerative diseases include, but are not limited to, Alzheimer's disease, Parkinson's disease, Huntington's disease, and amyotrophic lateral sclerosis. A preferred neurodegenerative disease for treatment is Alzheimer's disease. Alzheimer's diseases include familial, genetically associated, and sporadic AD. By “treatment” as used herein is intended prevention as well as therapy.

The term “subject” or “patient” as used herein is intended an animal, preferably a mammal, including a human. By “patient” is intended a subject in need of treatment of a neurodegenerative disease. The subject can express a neurodegenerative disease-linked mutation, as described above, such as a presenilin mutation.

In the embodiments of the invention as described herein, the agent can inhibit the CCE-reducing activity of the AD-linked mutation in the subject. The agent can inhibit γ-secretase activity in the subject.

The invention is also directed to a method of identifying a transient receptor potential protein (TRP) involved in increasing capacitative calcium entry (CCE) activity, the method comprising:

(a) providing cells which contain a presenilin mutation;

(b) overexpressing a TRP to be tested in the cells; and

(c) determining whether overexpression of the TRP increases CCE activity in the cells. The above described cells can be used in the invention.

SKF96365 is a CCE inhibitor, which has been found to potentiate γ-secretase activity. Thus, SKF96365 which has been, for example, radiolabeled, immunolabeled, or immobilized, can be used to identify cellular protein(s) which bind SKF96365 and are modified by treatment with SKF96365. The invention is directed to a method of identifying a cellular protein involved in capacitative calcium entry (CCE) inhibition, the method comprising:

(a) incubating cellular protein(s) and SKF96365; and

(b) characterizing and identifying the cellular protein(s) bound to the SKF96365.

For example, tritium [3H] labeled SKF96365 can be used to detect the cellular proteins in a binding assay. Samples can be prepared in buffer A (10 mM Na-HEPES, pH 7.4, 1.5 M KCl, 0.8 mM CaCl2, 10 mM ATP and 0.1-20 nM [3H]-SKF96365 in the presence or absence of 1 μM SKF96365 (non-radiolabeled). The membrane filters containing the sample can be incubated for 1 hour at 37° C. and assayed by autoradiography. If necessary, chromatographic fractions can be subjected to [3H]-SKF96365 binding assay. Similar experimental approaches have been published using other tritiated compounds (e.g. [3H]-ryanodine) (McPherson, P. S. et al. Neuron 7:17-25 (1991); Du, G. G. et al. J. Biol. Chem. 273:33259-33266 (1998)).

Alternatively, other CCE inhibitors can be used in the invention, such as, but not limited to, econazole, micozole, clotrimazole, and calmidazolium (Merritt, J. E. et al., Biochem. J. 271:515-522 (1990); Daly, J. W. et al., Biochem. Pharmacol 50:1187-1197 (1995)) plant alkaloids such as tetrandine, and hernandezine (Low, A. M. et al., Life Sci. 58:2327-2335 (1990)).

The cellular proteins can be obtained by from, for example, a cell extract prepared by methods well known in the art (Kim, T. -W. et al., J. Biol. Chem. 272:11006-11010 (1997)). The cellular protein bound to a CCE inhibitor can be characterized and identified by methods well known in the art, e.g., Western blotting, HPLC, FPLC, isolation of the protein, microsequencing of the protein, identification of the protein or its homologs in databases, and cloning of the gene encoding the protein of interest.

In the above embodiments of the invention, TRP activity can be measured in place of CCE activity using methods well known in the art, for example, as described in Ma, H. -T. et al., Science 287:1647-1651 (2000).

### Formulation and Methods of Administration

As used herein, “a pharmaceutically effective amount” is intended an amount effective to elicit a cellular response that is clinically significant, without excessive levels of side effects.

A pharmaceutical composition of the invention is thus provided comprising an agent useful for treatment of a neurodegenerative disease and a pharmaceutically acceptable carrier or excipient.

It will be desirable or necessary to introduce the pharmaceutical compositions directly or indirectly to the brain. Direct techniques usually involve placement of a drug delivery catheter into the host's ventricular system to bypass the blood-brain barrier. Indirect techniques, which are generally preferred, involve formulating the compositions to provide for drug latentiation by the conversion of hydrophilic drugs into lipid-soluble drugs. Latentiation is generally achieved through blocking of the hydroxyl, carboxyl, and primary amine groups present on the drug to render the drug more lipid-soluble and amenable to transportation across the blood-brain barrier. Alternatively, the delivery of hydrophilic drugs can be enhanced by intra-arterial infusion of hypertonic solutions which can transiently open the blood-brain barrier.

The blood-brain barrier (BBB) is a single layer of brain capillary endothelial cells that are bound together by tight junctions. The BBB excludes entry of many blood-borne molecules. In the invention, the agent can be modified for improved penetration of the blood-brain barrier using methods known in the art. Alternatively, a compound with increase permeability of the BBB can be administered to the subject. RMP-7, a synthetic peptidergic bradykinin agonist was reported to increase the permeability of the blood-brain barrier by opening the tight junctions between the endothelial cells of brain capillaries (Elliott, P. J. et al., Exptl. Neurol. 141:214-224 (1996)).

The invention further contemplates the use of prodrugs which are converted in vivo to the therapeutic compounds of the invention (Silverman, R. B., “The Organic Chemistry of Drug Design and Drug Action,” Academic Press, Ch. 8 (1992)). Such prodrugs can be used to alter the biodistribution (e.g., to allow compounds which would not typically cross the blood-brain barrier to cross the blood-brain barrier) or the pharmacokinetics of the therapeutic compound. For example, an anionic group, e.g., a sulfate or sulfonate, can be esterified, e.g., with a methyl group or a phenyl group, to yield a sulfate or sulfonate ester. When the sulfate or sulfonate ester is administered to a subject, the ester is cleaved, enzymatically or non-enzymatically, to reveal the anionic group. Such an ester can be cyclic, e.g., a cyclic sulfate or sultone, or two or more anionic moieties may be esterified through a linking group. In a preferred embodiment, the prodrug is a cyclic sulfate or sultone. An anionic group can be esterified with moieties (e.g., acyloxymethyl esters) which are cleaved to reveal an intermediate compound which subsequently decomposes to yield the active compound. In another embodiment, the prodrug is a reduced form of a sulfate or sulfonate, e.g., a thiol, which is oxidized in vivo to the therapeutic compound. Furthermore, an anionic moiety can be esterified to a group which is actively transported in vivo, or which is selectively taken up by target organs. The ester can be selected to allow specific targeting of the therapeutic moieties to particular organs, as described below for carrier moieties.

In yet another embodiment the therapeutic compounds or agents of the invention can be formulated to cross the blood-brain-barrier, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs thus providing targeted drug delivery (Ranade, J., Clin. Pharmacol. 29:685 (1989)). Exemplary targeting moieties include folate or biotin (U.S. Pat. No. 5,416,016), mannosides (Umezawa et al., Biochem. Biophys. Res. Comm. 53:103 8 (1988)), antibodies (Bloeman et al., FEBS Lett 357:140 (1995); Owais et al., Antimicrob. Agents Chemother. 39:180 (1995)), surfactant protein A receptor (Briscoe et al., Am. J. Physiol. 233:134 (1995)), gp 120 (Schreier et al., J. Biol. Chem. 269:9090 (1994); Killion and Fidler, Immunomethods 4:273 (1994)).

The pharmaceutical composition can be administered orally, nasally, parenterally, intrasystemically, intraperitoneally, topically (as by drops or transdermal patch), bucally, or as an oral or nasal spray. By “pharmaceutically acceptable carrier” is intended, but not limited to, a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

A pharmaceutical composition of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactant.

The compositions of the present invention can also contain adjuvants such as, but not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents such as sugars, sodium chloride, and the like. Prolonged absorption of the injectable pharmaceutical form can be brought about by the inclusion of agents which delay absorption such as aluminum monostearate and gelatin.

In some cases, in order to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are mixed with at least one item pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hardfilled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, can be prepared as a dry powder which can be pressurized or non-pressurized. In nonpressurized powder compositions, the active ingredients in finely divided form can be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

Alternatively, the composition can be pressurized and contain a compressed gas, such as nitrogen or a liquefied gas propellant. The liquefied propellant medium and indeed the total composition is preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

The compositions of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (see, for example, Prescott, Ed., Meth. Cell Biol. 14:33 et seq (1976)).

### Dosaging

One of ordinary skill will appreciate that effective amounts of the agents of the invention can be determined empirically and can be employed in pure form or, where such forms exist, in pharmaceutically acceptable salt, ester or prodrug form. The agents can be administered to a patient in need thereof as pharmaceutical compositions in combination with one or more pharmaceutically acceptable excipients. It will be understood that, when administered to a human patient, the total daily usage of the agents or composition of the present invention will be decided by the attending physician within the scope of sound medical judgement. The specific therapeutically effective dose level for any particular patient will depend upon a variety of factors:the type and degree of the cellular response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

For example, satisfactory results are obtained by oral administration of the compounds at dosages on the order of from 0.05 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 5 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging can also be arranged in a patient specific manner to provide a predetermined concentration of the agents in the blood, as determined by techniques accepted and routine in the art (HPLC is preferred). Thus patient dosaging can be adjusted to achieve regular on-going blood levels, as measured by HPLC, on the order of from 50 to 1000 ng/ml, preferably 150 to 500 ng/ml.

It will be readily apparent to one of ordinary skill in the relevant arts that other suitable modifications and adaptations to the methods and applications described herein can be made without departing from the scope of the invention or any embodiment thereof.

The following Example serves only to illustrate the invention, and is not to be construed as in any way to limit the invention.

### EXAMPLE

Since the Aβ42-promoting effect of FAD mutant presenilins does not appear to be cell type-specific (Scheuner, D., et al., Nature Med. 2:864-870 (1996); Borchelt, D. R, et al., Neuron 17:1005 (1996); Duff, K., et al., Nature 383:710 (1996); Citron, M., et al., Nature Med 3:67 (1996); Oyama, F., et al., J. Neurochem. 71:313 (1998)), multiple cell types were used, including SY5Y human neuroblastoma cells, CHO cells, and primary mouse neurons, to examine the effect of presenilin FAD mutations on a common Ca²⁺ regulatory pathway.

To examine the effect of a PS2 FAD mutation on CCE, stable SY5Y cell lines were established (Grudt, T. J., et al., Mol. Brain Res. 36:93 (1996)), harboring either wild-type or the Volga German FAD mutant (N1411) form of PS2 (FIG. 1A). In both native and vector-transfected SY5Y cells (FIG. 1A), PS2 was virtually undetectable in Western blots of straight lysate (Kim, T. -W., et al., J. Biol. Chem. 272:11006 (1997)). Protein quantitation, SDS-PAGE (14% or 4-20%), and Western blot analyses were performed (as described in Jayadev, S., J. Biol. Chem. 274:8261 (1999); Bezprozvanny, I., et al., Nature 351:751 (1991); Krause, K. -H., and Michalak, M., Cell 88:439 (1997); and Camacho, P. and Lechleiter, J. D., Cell 82:765 (1995)), indicating that detectable PS2 represents the transgene-derived protein variants in these stable cells. PS2 was detected mainly as endoproteolytic fragments in these cells, while full-length PS2 protein was detectable only after lengthy exposures (FIG. 1 A). As expected, transgene-derived PS2-CTF “replaced” endogenous PS1-CTF (FIG. 1A) (Thinakaran, G., et al., J. Biol. Chem. 272:28415 (1997)).

To induce CCE artificially, cells were incubated in Ca²⁺-free media containing an ER Ca²⁺ depleting reagent, such as cyclopiazonic acid (CPA), then washed and replenished with Ca²⁺-containing media. CCE was then monitored by ratiometric imaging using fura-2/AM (FIG. 1B) (Yoo, A. S. J., et al., Brain Res. 827:19 (1999)). Briefly, cells were grown on 25 mm-round glass coverslips for at least 24 hours before measuring [Ca²⁺]I. Fura-2/AM was dissolved in DMSO and further solubilized in Pluronic acid (0.08%), in HBSS (145 mM NaCl, 2.5 mM KCl, 1 mM MgCl₂, 20 mM HEPES, 10 mM glucose, and 1.8 mM CaCl₂ containing BSA (1%). When Ca²⁺-free medium was used, Ca²⁺ was replaced with 50 μM EGTA. Fura-2 acetoxymethyl ester (fura-2/AM) was loaded by incubation with HBSS containing fura-2/AM (5 μM) at 37° C. for 30 minutes. Fluorescence emission at 505 nm was monitored at 25° C. using a dual wavelength spectrofluorometer system with excitation at 340 and 380 nm. Ratios (fluorescence intensity at 340 nm/380 nm) were obtained from 8-frame averages of pixel intensities at each of the excitation wavelengths. When CCE was induced, Ca²⁺ influx was dramatically reduced in cells expressing N141I-PS2 as compared to either wild-type PS2 or vector alone (FIG. 1B). Although relative amplitudes of the CCE response were consistent among cells containing vector, wild-type PS2 or mutant PS2, the decay of Ca²⁺ influx was reproducibly increased in the presence of mutant PS2 as compared to the vector control. However, this effect was not seen in the presence of wild-type PS1. Multiple experiments were averaged to determine the mean peak amplitudes of CCE. In the N141I-PS2 cells, CCE was reduced by ˜58.5% compared to wild-type PS2-transfected cells (FIG. 1C).

It was determined whether FAD mutant presenilin-mediated downregulation of CCE also occurs in neurons. For this purpose, cultured primary neurons derived from transgenic mice harboring constructs encoding either wild-type or N141I FAD mutant forms of PS2 were utilized. As a source for these primary neuronal cultures, transgenic mice expressing wild-type or N141I FAD mutant forms of human PS2 under the transcriptional control of the PDGF promoter were generated. The genomic insertion and expression of human PS2 gene was confirmed by genotyping of tail DNA and RT-PCR of mRNA from brain tissues. To assess the expression of human PS2 protein in these transgenic animals, brain extracts of heterozygote animals expressing wild-type or N141I PS2 along with non-transgenic littermates were analyzed by combined immunoprecipitation -Western blot analyses using αPS2Loop (FIG. 8A). Elevated levels of PS2-CTF were observed in groups of transgenic mice expressing human wild-type PS2 and N141I-PS2 transgenes (FIG. 8A). In all PS2 founder transgenic mouse lines selected for the test, no detectable full-length PS2 polypeptides were observed. Founder lines with similar expression levels of PS2-CTF were selected for breeding and further use (FIG. 8B).

Cortical neuronal cultures were prepared from day 18.5 embryos of either heterozygote wild-type or N141I mutant PS2 animals. Embryos were plated in separate chambers and corresponding tissues were removed from each embryos and used for genotyping. In the neuronal cultures, non-neuronal cells were less than ˜10% and cell bodies of morphologically differentiated neurons were selected to conduct Ca²⁺ imaging experiments (n=˜50). CCE was dramatically suppressed in N141I-PS2 neurons as compared to wild-type PS2 neurons (FIG. 8C). Three independent Ca²⁺ imaging experiments were performed to determine mean peak amplitudes, indicating that ˜50% reduction of CCE in N141I-PS2 neurons as compared to wild-type PS2 neurons (FIG. 8D). Similar to what was observed in SY5Y cells, the amplitudes of CCE in neurons of wild-type animals were similar to that in neurons from non-transgenic animals.

To examine the effect of a PS1 FAD mutation on CCE, SY5Y cells stably transfected with the M146L PS1 FAD mutant (FIG. 4A) were used (Cruts, M. et al., Hum. Molec. Genet. 7:43-51 (1998)). When CCE was induced, the amplitude of the CCE response was markedly reduced in the M146L-PS1 cells (˜42.5% reduction) as compared to wild-type PS1-or γ-vector transfected cells (FIG. 1D and FIG. 1E). In addition to SY5Y cells, CCE was also found to be attenuated in CHO cells stably expressing M146L-PS1 as compared to wild-type PS1 (FIG. 1F). These data reveal that CCE was altered by both the M146L PS1 mutation and the N141I PS2 mutation, indicating that these separate FAD mutations both affect the cellular pathways involving CCE. Reduced CCE in the presence of PS FAD mutations also provides a potential mechanism underlying the decreased Ca²⁺ uptake observed in patient fibroblasts carrying a PS1 FAD mutation (Peterson, C., et al., New. Engl. J. Med. 312:1063 (1985)). IP₃-mediated intracellular Ca²⁺ release has been shown to be altered by the presence of PS FAD mutations in Xenopus oocytes (Guo, Q., et al., Neuroreport 8:379 (1996); Leissring, M. A., et al., J. Neurochem. 72:1061, (1999); Leissring, M. A., et al., J. Biol. Chem. 274:32535 (1999)). Direct interaction between the IP₃ receptor and a putative store-operated channel (i.e., TRP3) has recently been demonstrated (Kiselyov, K., et al., Mol. Cell 4:423 (1999)). To verify that this attenuation of the CCE response in PS1 and PS2 mutant cells was not simply due to the elevated levels of PS protein in our cell lines, CCE was measured in wild-type or mutant SY5Y cells with higher PS expression levels (as evidenced by accumulation of full-length PS protein in Western blots). It was found that varying levels of PS protein had no detectable effect on the CCE response. Although CCE potentiation by the TM Asp mutation was much greater in SY5Y cell lines (˜125%) as compared to CHO cell lines (˜40%) (FIG. 4D and FIG. 4E), the M146L-PS1 mutation affected CCE to a similar degree in both SY5Y and CHO cells (FIG. 1E and FIG. 1F).

To ensure that the FAD mutations were actually affecting CCE and not other types of Ca²⁺ influx, the effects of various pharmacological reagents in these cell lines were studied. The Ca²⁺ influx observed in all cell lines was blocked by pretreatment with the CCE inhibitors SKF96365 (Mason, M. J., et al., Am. J. Physiol. 264:C654 (1993)) and Calyculin A (CalyA) (Yao, Y., et al., Cell 98:475 (1999); Patterson, R. L., et al., Cell 98:487 (1999)) (FIG. 2A and FIG. 2C). However, nifedipine and ω-conotoxin GVIA, which inhibit L- and N-type Ca²⁺ channels, respectively, had virtually no effect on the Ca²⁺ influx observed (FIG. 2B); therefore, the alterations in[Ca²⁺]_{I}, were likely caused by modifications in CCE-specific Ca²⁺ influx. In addition, in the presence of nifedipine and ω-conotoxin GVIA, CCE was reduced in M146L cells to a similar extent as in untreated groups, suggesting that the mechanism underlying reduced CCE in mutant cells is independent of these types of voltage-operated Ca²⁺ channels.

It has previously been shown that disruption of the intracellular cytoskeleton by treatment with Cytochalasin D (CytoD)) impairs the IP₃-elicited release of Ca²⁺ from internal stores (Ribeiro, C. M. P., Jr., J. Biol. Chem. 272:26555 (1997)); in contrast, CytoD has no effect on CCE (Yao, Y., et al., Cell 98:475 (1999); Patterson, R. L., et al., Cell 98:487 (1999); Ribeiro, C. M. P., Jr., J. Biol. Chem. 272:26555 (1997)). Thus, it was examined whether CytoD could abolish the effect of PS FAD mutations on Ca²⁺ influx (FIG. 2D). CytoD had essentially no effect on Ca²⁺ influx in either wild-type PS1 or M146L-PS1 cells (FIG. 2D). Further, CCE was reduced in M146L-PS1 cells to a similar extent as in the untreated sets of experiments (FIG. 2D). Similar results were found using N141I-PS2 cells. These data indicate that FAD-associated presenilin mutations may directly affect CCE independent of the Ca²⁺ mobilization pathways that require an intact cytoskeleton (Ribeiro, C. M. P., Jr., J. Biol. Chem. 272:26555 (1997)).

Functional activities of putative plasma membrane CCE channels can be detected as calcium release activated Ca²⁺ current, also known as store-operated Ca²⁺ current (I_{CRAC}) (Hoth, M. and Penner, R, Nature 355:353 (1992); Zweifach, A. and Lewis, R. S., Proc. Natl. Acad. Sci. USA. 90:6295 (1993)). The effects of presenilin FAD mutations on CCE were further investigated by examining I_{CRAC} in wild-type and M146L-PS1 CHO cells. The time course of activation of I_{CRAC} was determined in single cells followed by passive store depletion via patch pipettes containing Ca²⁺-chelating reagent BAPTA in the whole cell configuration and Na²⁺ was used as the charge carrier (Kerschbaum, H. H. and Cahalan, M. D., Science 283:836 (1999)). The currents were activated slowly under this condition and reached the maximal level in ˜5 min in the wild-type PS1 cells after establishment of whole cell configuration (FIG. 9A and FIG. 9B). In contrast, M146L-PS1 cells exhibited severely impaired I_{CRAC} (FIG. 9A and FIG. 9B). Similar data have been obtained using stable M146L-PS1 SY5Y cell lines. The average current density was significantly reduced in M146L-PS1 CHO cells as compared to wild-type cells (FIG. 9C). Under our experimental conditions, pretreatment of cells with SKF96365 virtually eliminated I_{CRAC} in wild-type PS1 CHO cells (FIG. 9C), indicating that I_{CRAC} is sensitive to pre-treating cells with SKF96365.

A novel arachidonate-regulated current (I_{ARC}) has been reported and channel properties of I_{ARC} appeared to be similar to that of I_{CRAC} (Shuttleworth, T. J., J. Biol. Chem. 271:21720 (1996)). However, I_{ARC} is activated even after the store depletion (Mignen, O. and Shuttleworth, T. J., J. Biol. Chem. 275:9114 (2000)). It was determined whether PS1 FAD mutation affects I_{ARC} after the induction of I_{CRAC} via store depletion. Aracidonic acid-induced currents followed by I_{CRAC} were preserved in both wild-type and M146L-PS1 cells (FIG. 9D). This indicates that presenilin FAD specifically affects the store-dependent current, I_{CRAC}, but not store-independent currents such as I^{ARC}.

It has recently been demonstrated that a deficiency in PS1 abrogates the γ-secretase-mediated cleavage of APP and the subsequent generation of Aβ (De Strooper, B., et al., Nature 391:387 (1998)). In addition, PS1 deficient neurons exhibit abnormal trafficking of select membrane proteins, including Notch and TrkB (Annaert, W., and De Strooper, B., Trends Neurosci. 22:439 (1999); Naruse, S., et al., Neuron 21:1213 (1998); De Strooper. B., et al., Nature 398:518 (1999); Struhl, G., and Greenwald, I., Nature 398:522 (1999); Ye, Y., et al., Nature 398:525 (1999); and Steiner, H., et al., J. Biol. Chem. 274:28669 (1999)). To examine the effect of a PS1 deficiency on CCE, cortical neuronal cell cultures derived from day 15.5 embryos of either heterozygote PS1 +/−, homozygote PS1+/+, or PS−/−transgenic mice were utilized (Shen, J., et al., Cell 89:629 (1997)).

Primary cell cultures were prepared as described in Roth, K. A., et al., J. Neurosci. 16:1753 (1996). Briefly, pregnant mice were sacrificed on EI 5.5. The morning of the day the vaginal plugs were observed was designated as E0.5. The uterus was removed under sterile conditions and the embryos were rapidly transferred to HBSS dissociation media. The dissociation media contained IX HBSS (Gibco, Grand Island, N.Y.), 15 mM Hepes, 7.5% sodium bicarbonate, 2.47 g/0.5 L glucose, pH 7.4. The tails were harvested for DNA extraction and PCR analysis of genotype. The brain was dissected out of the head with forceps and the pia and connective tissue were carefully removed. After dissection was complete, brains were washed with fresh HBSS dissociation media and the tissue was transferred to a 15 ml falcon tube containing 1 ml trypsin and 0.001 % DNase. Tubes were placed in a 37° C. water bath for 10-12 minutes, shaking every 2-3 minutes to break the clump of tissues. 1.5 ml of neurobasal media with 10% serum was added to each of the tubes. Cell were mildly dissociated using a polished Pasteur pipette. Tissues are allowed to settle at room temperature for 4-6 minutes. Supernatant was removed and spun for 5 min at room temperature at 1000 rpm, and the pellet was resuspend in 2 ml neurobasal media with serum. Cell s were counted and plated at a density of 40,000 cells/cm². Cell s were plated onto 25 mm coverslips coated with poly-L-lysine (0.25 mg/ml). After 2 hrs, media were removed and replaced with neurobasal media supplemented with B27, glutamine and Pen/Strep. Cultured neurons used for Ca²⁺ imaging were also analyzed in parallel by Western blots to verify the PS1 deficiency in PS1−/−neurons (FIG. 3A). A dramatic increase in CCE was observed in PS1-deficient neurons as compared to control neurons (FIG. 3B). These findings indicate that CCE is greatly potentiated by the absence of PS1, suggesting that PS1 may play an inhibitory role in the CCE response.

To define further the mechanism underlying the enhanced CCE in PS1-deficient neurons, the effect of inhibition of PS1-associated γ-secretase activity on CCE was examined. Abrogation of γ-secretase activity is one of the key phenotypic features of PS1 deficient cells (De Strooper, B., et al., Nature 391:387 (1998)). Recently, it was found that two PS1 transmembrane aspartate residues, D257 and D385, are critical for PS1-associated γ-secretase activity as well as PS1 endoproteolysis (Wolfe, M. S., et al., Nature 398:513 (1999)). Stably overexpressing the inactive PS1 D257A variant has been shown to inhibit PS1-associated γ-secretase activity (Wolfe, M. S., et al., Nature 398:513 (1999)).

SY5Y cell lines stably expressing a PS1 variant containing a TM aspartate mutation that was shown to abrogate the biological activities of PS1 (D257A-PS1) 1 was established (FIG. 4A). In these cells, the impaired endoproteolytic processing of PS1 resulted in the accumulation of full-length PS1 holoprotein which largely replaced the endogenous PS1 C-terminal fragment (FIG. 4A). An increased accumulation of endogenous APP C-terminal fragments (APP-CT83) was observed (FIG. 4A), although the level of APP-CT83 was not as robust as in a previous study, which utilized APP-overexpressing cells (Wolfe, M. S., et al., Nature 398:513 (1999)). Interestingly, CCE was enhanced by ˜125% in D257A-PS1 cells as compared to wild-type PS1 or FAD mutant PS1 SY5Y cells (FIG. 4B and FIG. 4C). CCE was also potentiated by two separate TM aspartate mutations (D257A and D385A) in stable CHO cell lines (FIG. 4D). These data reveal that mutating the TM Asp residues, both of which have been shown to abolish the biological activities of PS1, dramatically potentiates CCE. In addition, these results indicate that CCE activity is inversely correlated to presenilin-linked γ-secretase activity.

To gain insight into the molecular link between the PS FAD-driven changes in the CCE response and alterations in Aβ42 production, the effect of direct CCE inhibition on the generation of Aβ42 was examined. Specifically, the effect of the CCE antagonist SKF96365 on Aβ production was studied using a sensitive Aβ-specific sandwich ELISA (Xia, X., et al., J. Biol. Chem. 272:7977 (1997)). SKF96365 decreased both store depletion-activated Ca²⁺ influx and currents (FIG. 9C). Since Aβ levels (e.g. Aβ42) in SY5Y cells are not readily detectable, CHO or 293 cells stably overproducing human APP695 were utilized. SKF96365 has been shown to have a minor inhibitory effect on voltage-operated Ca 2+ channels (Merritt, J. E. et al., Biochem. J. 271:515 (1990); Mason, M. J., et al., Am. J. Physiol. 264:C564 (1993); Grundt, T. J., et al., Mol. Brain Res. 36:93 (1996)); therefore, nifedipine and ω-conotoxin GVIA were included as negative controls to ensure the CCE-specificity of SKF96365 on Aβ generation.

Interestingly, treatment of CHO or 293 cells stably expressing human APP with SKF96365 specifically elevated the ratio of Aβ42/Aβ total (FIG. 5A and FIG. 5B). This Aβ42-promoting effect of SKF96365 was dose-dependent (FIG. 5C) and inversely correlated with relative magnitudes of CCE (FIG. 5D). Under these conditions, SKF96365 treatment did not alter secreted APP-α levels or cell viability in these cultures.

To detect secreted APP produced from the γ-secretase-mediated cleavage of APP (APPs-α), media collected from the above-mentioned samples of 293 cells were immunoprecipitated by antibody 22C11 and blotted with antibody 6E10. In contrast, nifedipine and ω-conotoxin GVIA had no significant effect on Aβ42 generation (FIG. 5A and FIG. 5B). The concentrations of SKF96365, nifedipine and ω-conotoxin GVIA were similar to those used in other studies (Grudt, T. J. et al., Mol. Brain Res. 36:93 (1996); Merritt, J. E. et al., Biochem. J. 271:515 (1990); Vazquez, G., et al., J. Biol. Chem. 273:33954 (1998); Jayadev, S., et al., J. Biol. Chem. 274:8261 (1999)). These results demonstrate that inhibition of the cellular pathways involving CCE specifically increases Aβ42, which is a molecular phenotype linked with FAD mutant presenilins.

It was determined whether the Aβ42-elevating effect of SKF96365 requires the biological activity of the presenilins. For this purpose, CHO cells stably expressing D257A-PS1 were treated with SKF96365 and Aβ generation was measured. As previously reported (Wolfe, M. S. et al., Nature 398:513 (1999)), total Aβ levels were dramatically lower in D257A-PS1 cells than wild-type PS1-expressing CHO cells (FIG. 5E). Aβ42 was also reduced in D257A-PS1 cells relative to wild-type PS1 cells, but to a lesser extent than total Aβ (FIG. 5F). Treatment with SKF96365 did not restore the generation of either total Aβ or Aβ42 in the D257A-PS1 cells, indicating that the biological activity of PS1 is required for the Aβ42-promoting effect of SKF96365. In D257A cells, relative Aβ42 levels following treatment with SKF96365 was greater than 90% of total Aβ levels (FIG. 5E and FIG. 5F). Under identical conditions (50 μM SKF96365, 12 hrs), the degree of CCE reduction in D257A-PS1 cells was much less as compared to wild-type PS1 cells reduction.

It was determined whether CCE reduction by FAD-linked presenilin mutations is simply due to the increased accumulation of Aβ42 inside or outside of the cells. Utilizing CHO-APP cells that produce substantially elevated levels of APP and Aβ42 (Xia, X., et al., J. Biol. Chem. 272:7977 (1997)), it was found that stable overproduction of APP (and the subsequent increase in Aβ42) had virtually no effect on CCE (FIG. 6A). Furthermore, pre-treatment of the cells with Aβ42 also had no detectable effect on the CCE response (FIG. 6A vs. FIG. 6B). Similar data have been obtained using 293 and SY5Y cell lines. The Aβ42 peptides were obtained from Bachem and dissolved in PBS at 1 mg/ml directly before use. Cell viability was not affected under these conditions. These findings suggest that reduced CCE in FAD mutant presenilin cells is not simply due to increased extracellular or intracellular levels of Aβ42.

Expression and detection of TRP1 and TRP3 in CHO cells are shown in FIG. 7A. Stable CHO cell lines expressing either wild-type PS1(W) or M146L mutant PS1 (M) were transiently transfected with empty vector (Control), FLAG-tagged TRP1 expression construct (TRP1-FLAG) (Kim, T. -W. et al., J. Biol. Chem. 272:11006-11010 (1997)), and MYC-tagged TRP3 expression construct (TRP3-MYC) (Evans, G. I. et al., Mol. Cell. Biol. 5:3610-3616 (1985)). The cell lysates were analyzed by Western blot analyses using anti-FLAG (left) or anti-MYC (right) antibodies.

Effect of overexpression of TRP 1 and TRP3 on capacitative calcium entry (CCE) in stable CHO cells expressing M146L FAD mutant PS1 is shown in FIG. 7B. CCE was potentiated in both TRP1-and TRP3-transfected cells as compared to vector-transfected (Control) cells, but to greater extent in TRP3-expressing cells. The ratiometric calcium imaging was performed as described above.

Effects of overexpression of vector, TRP1, and TRP3 on the ratio of Aβ42/Aβtotal in CHO cells stably expressing M146L mutant PS1 are shown in FIG. 7C. Amounts of Aβ42 and Aβtotal were determined by sandwich ELISA as described above. Overexpression of TRP3 decreased the ratio of Aβ42/Aβtotal .

In summary, these studies reveal a connection between presenilin FAD mutations and CCE; specifically, CCE is universally reduced in the presence of such mutations. Moreover, reduced CCE led to increased production of Aβ42, while increased Aβ levels had no apparent effect on CCE. These findings suggest that reduced CCE may be an important molecular event associated with FAD neuropathogenesis. In contrast, CCE was potentiated by the absence of PS1 or by expression of the artificial D257A-PS1 variant, which has an inhibitory effect on γ-secretase activity; these results reveal an intimate interaction between γ-secretase activity and the CCE pathway. These data prompted the interesting possibility that reduced CCE is an early pathogenic effect of presenilin FAD mutants, preceding increases in Aβ42. Augmentation of CCE could therefore potentially be employed to reduce PS-associated γ-secretase activity, by identifying agonists of plasma membrane store-operated Ca²⁺ channels (e.g., TRP) that mediate CCE (Bimbaumer, L., et al., Proc. Natl. Acad. Sci. USA 93:15195 (1996); Zhu, X., et al., Cell 85:661 (1996)).

### Conclusion

The presence of biologically active PS1 or PS2 is essential for the generation of Aβ through γ-secretase cleavage of APP (De Strooper, B., et al., Nature 391:387 (1998)). The current studies demonstrated that abrogation of biological activities of PS1, by either knocking out PS1 or expressing inactive PS1 mutants, greatly potentiated CCE, suggesting that a normal function of PS1 (and perhaps PS2) is to modulate CCE. We also showed that treating cells with a CCE inhibitor (SKF96365) downregulates CCE and I_{CRAC} and selectively elevates Aβ42 generation. However, increased cellular levels of Aβ42 had no effect on CCE, suggesting that reduced CCE might be an early cellular event leading to the increased Aβ42 generation associated with presenilin FAD mutations. Interestingly, preliminary data revealed that direct inhibition of Aβ generation using a synthetic γ-secretase inhibitor displayed no effects on CCE, further supporting the idea that presenilin-mediated modulation of CCE is an upstream event of Aβ generation.

According to our model, autosomal dominant FAD mutant presenilins exert a gain of function by downregulating CCE while increasing IP₃-mediated release from the ER store, leading to diminished luminal Ca²⁺ concentration ([Ca²⁺]_{ER}) (Waldron, R. T., et al., J. Biol. Chem. 272:6440 (1997); Hofer, A. M., et al., J. Cell. Biol. 140:325 (1998)). It is interesting to note that changes in [Ca²⁺]_{ER} influence a number of cellular functions including chaperone activities and gene expression (reviewed in Meldolesi, J., and Pozzan, T., TIBS 23:10 (1998)). Therefore, it is tempting to speculate that reduced CCE may also be an upstream event leading to other molecular phenotypes associated with FAD mutant presenilins, including altered unfolded protein response (Niwa, M., et al., Cell 99:691 (1999); Katayama, T., et al., Nat. Cell. Biol. 1:479 (1999)) and increased vulnerability to apoptotic stimuli (Wolozin, B., et al., Science 274:1710 (1996); Deng, G., et al., FEBS Lett. 397:50 (1996); Janicki, S., and Monteiro, M. J., J. Cell Biol. 139:485 (1997)). Interestingly, in transgenic mice harboring spinocerebellar ataxia type 1 (SCA1) mutant gene products, TRP3, SERCA2, and IP₃-R, all components of CCE, were specifically downregulated. This suggests the potential contribution of CCE dysregulation in other neurodegenerative diseases in addition to AD (Lin, X., et al., Nature Neurosci. 3:157 (2000)).

CCE involves direct physical interaction between the ER and plasma membrane constituents (reviewed in Putney, J. W., Jr., Cell 99:5 (1999a); Berridge, M. J., et al., Science 287:1604 (2000)). According to this conformational coupling mechanism, a conformational change of the IP₃ receptor (IP₃-R) upon agonist stimulation and subsequent release of Ca²⁺ leads to the formation of a molecular complex containing IP₃-R bound to molecular constituents in the plasma membrane harboring CCE channels. This then allows extracellular Ca²⁺ to replenish the ER store (Kiselyov, K., et al., Nature 396:478 (1998); Kiselyov, K., et al., Mol. Cell 4:423 (1999); Bouray, G., et al., Proc. Natl. Acad. Sci. USA 96:14955 (1999); Putney, J. W., Jr., Cell 99:5 (1999a)). It has been postulated that the presenilins modulate the γ-secretase activity via few possible mechanisms: the presenilins might be the γ-secretases themselves, serve as essential cofactors for the γ-secretase action, or regulate intracellular trafficking of a putative γ-secretase to the target site where relevant substrates are localized (De Strooper, B., et al., Nature 391:387 (1998); Wolfe, M. S., et al., Nature 398:513 (1999); Naruse, S., et al., Neuron 21:1213 (1998); reviewed in Selkoe, D. J., Curr. Opin. Neurobiol. 10:50 (2000)). Given a role for presenilins in governing CCE, the presenilins may also modulate proteolytic processing of APP and Notch at or near the cell surface (Annaert, W., and De Strooper, B., Trends Neurosci. 22:439 (1999)) at sites of ER-plasma membrane coupling. It is conceivable that the presenilins may also regulate the cleavage of protein(s) involved in modulating CCE. In any event, a gain in the biological activity of the presenilins, owing to autosomal dominant FAD mutations, may attenuate CCE while increasing γ-secretase activity. Further experimentation will be necessary to elucidate this connection. Finally, augmentation of CCE, through the identification of agonists of plasma membrane store-operated Ca²⁺ channels (e.g. TRP or as yet undiscovered CCE channels) that mediate CCE (Birnbaumer, L., Proc. Natl. Acad. Sci. USA 93:15195 (1996); Zhu, X., Cell 85:661 (1996); Putney, J. W., Jr., Proc. Natl. Acad. Sci. USA 96:14669 (1999b); Li, H. -S., et al., Neuron 24:261 (1999); Philipp, S., In Calcium Signaling, J. W. Putney, Jr., eds. (Boca Raton, Fla.: CRC Press), pp. 321-342. (2000)), could potentially be employed to reduce PS-associated γ-secretase activity, and the generation of Ab as a novel therapeutic means for preventing or treating AD.

What is claimed is:

1. A method of identifying an agent useful in treatment of a neurodegenerative disease, said method comprising:
(a) assaying for capacitative calcium entry (CCE) activity in cells treated with an agent;
(b) assaying for CCE activity in cells untreated with said agent; and
(c) comparing the CCE activities of (a) and (b) to determine whether said agent potentiates CCE activity in said cells treated with said agent, thereby identifying an agent useful in treatment of a neurodegenerative disease.

2. The method of claim 1, wherein said cells have a neurodegenerative disease-linked mutation.

3. The method of claim 2, wherein said neurodegenerative disease-linked mutation is a mutation causative of a neurodegenerative disease selected from Alzheimer's disease, Parkinson's disease, Huntington's disease and amyotrophic lateral sclerosis.

4. The method of claim 1, wherein said cells express an Alzheimer's disease-linked presenilin mutation.

5. The method of claim 1, wherein said cells contain an amyloid β-protein precursor (APP) mutant.

6. The method of claim 1, wherein said cells contain an apolipoprotein E (APOE) mutant.

7. The method of claim 1, wherein said neurodegenerative disease is selected from Alzheimer's disease, Parkinson's disease, Huntington's disease and amyotrophic lateral sclerosis.

8. The method of claim 7, wherein said agent is useful in treatment of Alzheimer's disease and inhibits the CCE-reducing activity of the Alzheimer's disease-linked mutation.

9. The method of claim 1, further comprising:
(d) assaying for CCE activity in cells treated with said agent, wherein said cells overexpress a transient receptor potential protein (TRP);
(e) assaying for CCE activity in cells treated with said agent, wherein said cells do not overexpress a TRP; and
(f) comparing the CCE activities of (d) and (e) to determine whether said agent potentiates CCE activity in said cells that overexpress a TRP.

10. A method of identifying an agent which inhibits capacitative calcium entry (CCE)-linked γ-secretase activity, said method comprising:
(a) assaying for CCE activity in cells treated with an agent;
(b) assaying for CCE activity in cells untreated with said agent; and
(c) comparing the CCE activities of (a) and (b) to determine whether said agent increases CCE activity in said cells treated with said agent, thereby identifying an agent which inhibits γ-secretase activity.

11. The method of claim 10, wherein said cells express an Alzheimer's disease-linked mutation.

12. The method of claim 10, wherein said cells express an Alzheimer's disease-linked presenilin mutation.

13. The method of claim 10, wherein said cells contain an amyloid β-protein precursor (APP) mutation.

14. The method of claim 10, wherein said cells contain an apolipoprotein E (APOE) mutation.

15. The method of claim 10, wherein said agent useful in treatment of AD inhibits the CCE-reducing activity of the Alzheimer's disease-linked presenilin mutation.

16. The method of claim 10, further comprising:
(d) assaying for CCE activity in cells treated with said agent, wherein said cells overexpress a transient receptor potential protein (TRP);
(e) assaying for CCE activity in cells treated with said agent, wherein said cells do not overexpress a TRP; and
(f) comparing the CCE activities of (d) and (e) to determine whether said agent potentiates CCE activity in said cells that overexpress a TRP.

17. A method of treatment of a neurodegenerative disease in a subject, said method comprising:administering to said subject a pharmaceutically effective amount of an agent capable of potentiating capacitative calcium entry (CCE) activity in said subject.

18. The method of claim 17, wherein said neurodegenerative disease is selected from the group consisting of Alzheimer's disease, Parkinson's disease, Huntington's disease and amyotrophic lateral sclerosis.

19. The method of claim 18, wherein said neurodegenerative disease is Alzheimer's disease.

20. The method of claim 19, wherein said subject expresses a Alzheimer's disease-linked presenilin mutation.

21. The method of claim 20, wherein said agent inhibits the CCE-reducing activity of the Alzheimer's disease-linked presenilin mutation in said subject.

22. The method of claim 19, wherein said agent inhibits γ-secretase activity in said subject.

23. A method of identifying a transient receptor potential protein (TRP) involved in increasing capacitative calcium entry (CCE) activity, said method comprising:
(a) providing cells which contain a presenilin mutation;
(b) overexpressing a TRP to be tested in said cells; and
(c) determining whether overexpression of said TRP increases CCE activity in said cells.

24. A method of identifying cellular components involved in capacitative calcium entry (CCE) inhibition, said method comprising:
(a) incubating cellular protein(s) and SKF96365; and
(b) characterizing and identifying the cellular protein(s) bound to said SKF96365.

25. A method of identifying an agent that reduces the ratio of amyloid β peptide Aβ42 to total amyloid β protein, reduces amyloid β peptide Aβ42 levels, or reduces the production of amyloid β peptide Aβ42 in a cell or extracellular medium, comprising:
(a) assaying for capacitative calcium entry (CCE) activity in cells treated with an agent;
(b) assaying for CCE activity in cells untreated with said agent;
(c) comparing the CCE activities of (a) and (b) to determine whether said agent potentiates CCE activity in cells treated with said agent, thereby identifying an agent that reduces the ratio of amyloid β peptide Aβ42 to total amyloid β protein, reduces amyloid β peptide Aβ42 levels, or reduces the production of amyloid β peptide Aβ42 in a cell or extracelluar medium.

26. The method of claim 25, wherein the cells comprise nucleic acid encoding an Alzheimer's disease-linked mutation.

27. The method of claim 25, wherein the cells comprise nucleic acid encoding a mutant presenilin.

28. The method of claim 25, wherein the cells comprise nucleic acid encoding a mutant amyloid β protein precursor.

29. A method of identifying an agent that reduces the ratio of amyloid β peptide Aβ42 to total amyloid β protein, reduces amyloid β peptide Aβ42 levels, or reduces the production of amyloid β peptide Aβ42 in a cell or extracellular medium, comprising:
(a) assaying for transient receptor potential protein (TRP) activity in cells treated with an agent;
(b) assaying for TRP activity in cells untreated with said agent;
(c) comparing the TRP activities in (a) and (b) to determine whether said agent potentiates TRP activity in cells treated with said agent, thereby identifying an agent that reduces the ratio of amyloid β peptide Aβ42 to total amyloid β protein, reduces amyloid β peptide Aβ42 levels, or reduces the production of amyloid β peptide Aβ42 in a cell or extracellular medium.

30. A method of identifying a candidate agent useful in treatment of a neurodegenerative disease, comprising:
(a) assaying for transient receptor potential protein (TRP) activity in cells treated with an agent;
(b) assaying for TRP activity in cells untreated with said agent;
(c) comparing the TRP activities of (a) and (b) to determine whether said agent potentiates TRP activity in cells treated with said agent, thereby identifying a candidate agent useful in treatment of a neurodegenerative disease.

31. A method of reducing the ratio of amyloid β peptide Aβ42 to total amyloid β protein, reducing amyloid β peptide Aβ42 levels, or reducing the production of amyloid β peptide Aβ42 in a cell or extracellular medium, comprising administering to said cell or extracellular medium an agent that potentiates CCE activity in cells, thereby reducing the ratio of amyloid β peptide Aβ42 to total amyloid β protein, reducing amyloid β peptide Aβ42 levels, or reducing the production of amyloid β peptide Aβ42 in said cell or extracellular medium.

32. A method of reducing amyloid β peptide Aβ42 levels in a cell or extracellular medium, comprising administering to said cell or extracellular medium an agent that potentiates CCE activity in cells, thereby reducing amyloid β peptide Aβ42 levels in said cell or extracellular medium.

33. A method of reducing the production of amyloid β peptide Aβ42 in a cell or extracellular medium, comprising administering to said cell or extracellular medium an agent that potentiates CCE activity in cells, thereby reducing the production of amyloid β peptide Aβ42 in said cell or extracellular medium.

34. A method of treating a neurodegenerative disease in a subject, comprising administering to the subject a pharmaceutically effective amount of an agonist of a store-operated calcium channel.

35. A method of treating a neurodegenerative disease in a subject, comprising administering to the subject a pharmaceutically effective amount of an agonist of a transient receptor potential protein (TRP).

36. A method of treating a neurodegenerative disease in a subject, comprising administering to the subject a pharmaceutically effective amount of an agent that increases the level of transient receptor potential protein (TRP) in the subject.

37. A method of reducing the ratio of amyloid β peptide Aβ42 to total amyloid β protein, reducing amyloid β peptide Aβ42 levels, or reducing the production of amyloid β peptide Aβ42 in a cell or extracellular medium, comprising administering to s aid cell or extracellular medium an agonist of a transient receptor potential protein (TRP), thereby reducing the ratio of amyloid β peptide Aβ42 to total amyloid β protein, reducing amyloid β peptide Aβ42 levels, or reducing the production of amyloid β peptide Aβ42 in said cell or extracellular medium.

38. A method of reducing the ratio of amyloid β peptide Aβ42 to total amyloid β protein, reducing amyloid β peptide Aβ42 levels, or reducing the production of amyloid β peptide Aβ42 in a cell or extracellular medium, comprising administering to said cell or extracellular medium an agent that regulates expression of a transient receptor potential protein (TRP), thereby reducing the ratio of amyloid β peptide Aβ42 to total amyloid β protein, reducing amyloid β peptide Aβ42 levels, or reducing the production of amyloid β peptide Aβ42 in said cell or extracellular medium.

39. A method of reducing the ratio of amyloid β peptide Aβ42 to total amyloid β protein, reducing amyloid β peptide Aβ42 levels, or reducing the production of amyloid β peptide Aβ42 in a cell or extracellular medium, comprising administering to said cell or extracellular medium an agent that regulates cellular maturation of a transient receptor potential protein (TRP), thereby reducing the ratio of amyloid β peptide Aβ42 to total amyloid β protein, reducing amyloid β peptide Aβ42 levels, or reducing the production of amyloid β peptide Aβ42 in said cell or extracellular medium.

40. A method of reducing the ratio of amyloid β peptide Aβ42 to total amyloid β protein, reducing amyloid β peptide Aβ42 levels, or reducing the production of amyloid β peptide Aβ42 in a cell or extracellular medium, comprising increasing the level of transient receptor potential protein (TRP) in said cell or extracellular medium, thereby reducing the ratio of amyloid β peptide Aβ42 to total amyloid β protein, reducing amyloid β peptide Aβ42 levels, or reducing the production of amyloid β peptide Aβ42 in said cell or extracellular medium.

41. A method of identifying an agent useful in treatment of a neurodegenerative disease, said method comprising:
(a) assaying for capacitative calcium entry (CCE) activity in cells treated with an agent, wherein the cells overexpress a transient receptor potential protein (TRP);
(b) assaying for CCE activity in cells treated with said agent, wherein the cells do not overexpress a TRP; and
(c) comparing the CCE activities of (a) and (b) to determine whether said agent potentiates CCE activity in the cells that overexpress a TRP.
